(19) United States
(12) Patent Application Publication
(10) Pub. No.: US 2013/0157283 A1
(43) Pub. Date: Jun. 20, 2013

(54) RAPID PATHOGEN DIAGNOSTIC DEVICE AND METHOD

(71) Applicants: Chong Wing Yung, Boston, MA (US); Donald E. Ingber, Boston, MA (US); Ryan Mcomber Cooper, Cambridge, MA (US); Frank Vollmer, Erlangen (DE); Karel Domansky, Charlestown, MA (US); Daniel Christopher Leslie, Brookline, MA (US); Michael Super, Lexington, MA (US)

(72) Inventors: Chong Wing Yung, Boston, MA (US); Donald E. Ingber, Boston, MA (US); Ryan Mcomber Cooper, Cambridge, MA (US); Frank Vollmer, Erlangen (DE); Karel Domansky, Charlestown, MA (US); Daniel Christopher Leslie, Brookline, MA (US); Michael Super, Lexington, MA (US)

(73) Assignee: PRESIDENT AND FELLOWS OF HARVARD COLLEGE, Cambridge, MA (US)

(21) Appl. No.: 13/522,800

(22) PCT Filed: Jan. 19, 2011
(86) PCT No.: PCT/US2011/021718

§ 371 Date: Mar. 8, 2013

Related U.S. Application Data
(60) Provisional application No. 61/296,357, filed on Jan. 19, 2010.
(60) Provisional application No. 61/296,355, filed on Jan. 19, 2010.

(51) Int. Cl. C12Q 1/04

(52) U.S. Cl. CPC C12Q 1/04
USPC 435/7.1; 435/287.3

(57) ABSTRACT

A microfluidic device of a diagnostic and detection system includes an inlet port connected by one or more microchannels to an outlet port and includes a capture and visualization chamber (CVC) connected to at least one microchannel. A fluid to be analyzed can be mixed with magnetic microbeads that have an affinity to become bound to target components, such as pathogens in the fluid. The fluid including the magnetically bound target components can be injected through the microfluidic device. Magnetic field gradient, such as provided by permanent or electro-magnets, can be applied to the fluid and the magnetically bound target components flowing through the microfluidic device to cause the magnetically bound target components to migrate into the (CVC) and become separated from the fluid. The magnetically bound target components can be analyzed and tested using various techniques to detect the presence of specific organic and inorganic materials, such as pathogens in bio-fluids and contamination in liquid food sources (e.g. water). The device and method provide a system for rapidly detecting pathogens and contamination in relatively small fluid samples.

### RELATED APPLICATIONS

This application claims benefit under 35 U.S.C. §119(e) of the U.S. Provisional Application No. 61/296,355, filed Jan. 19, 2010 and No. 61/296,357, filed Jan. 19, 2010, the contents of both of which are incorporated herein by reference in their entirety.

### GOVERNMENT SUPPORT

This invention was made with government support under grant no. W81XWH-07-2-0011 and no. W81XWH-05-1-0115 W8 awarded by U.S. Department of Defense. The government has certain rights in the invention.

### FIELD OF THE INVENTION

The present invention relates to molecular immunology, microbial pathogens, and systems and methods for detecting and/or removing pathogens in blood. More specifically, the present invention provides a device and method for rapid pathogen diagnosis of patients with infectious diseases, blood-borne infections, or sepsis.

### BACKGROUND OF THE INVENTION

The speed of pathogen diagnosis in a patient with a microbial infection can mean the difference between life and death. However, it is difficult to diagnose the existence of a serious infection because the presence of living pathogens (e.g., bacteria, fungi, viruses or protozoans) in tissues or the blood, and the systemic inflammatory and immune response to infection, known as sepsis, often have similar generic symptoms, including fever, chills, rigor, increased heart and respiratory rates, and changes in white blood cell counts at early stages of the disease. Samples of biological fluids (e.g., blood, urine, cerebrospinal fluid, sputum, tracheal aspirates, feces) obtained from patients can be examined under a microscope when an infection is suspected to glean some information about the nature of the infectious source. However, due to low numbers, other components (cellular, molecular, mucus, etc.) in these samples, and the reality that viruses and some bacteria can not be detected with conventional staining techniques (e.g., gram or acid-fast stains), negative results can not be interpreted as disproving the existence of infectious pathogens in the sample. Due to the low incidence of pathogens in blood (<100 pathogen cells/mL), even in patients with late stage sepsis, existing direct staining methods are not useful for pathogen identification in this condition.

The preferred, but slowest, method for detection of a microbial infection is to culture biological fluids from the suspected source of infection (e.g., blood, sputum, tracheal aspirates, cerebrospinal fluid, urine, etc.), which is commonly carried out only in a hospital or commercial clinical microbiology laboratory setting. Liquid cultures can permit detection of the general existence of growing organisms in the fluid, but then the organism must be transferred to other growth media (e.g., agar plates) to identify the specific species of the pathogen, and to carry out sensitivity testing to determine their relative response to various potential antibiotic therapies. Importantly, not all pathogens can be easily cultured, and some can not be cultured at all. Moreover, no current point-of-care (POC) methods exist for community or emergency physicians to identify patients with early systemic infection due to the presence of blood-borne pathogens who require immediate transfer to a hospital setting (where a full workup can be carried out and intravenous therapy can be administered) from those who will have a self-limited local infection or one that will respond to conventional oral therapy.

Wide-spectrum, intravenous, anti-bacterial antibiotics may be administered prophylactically at the first suspicion of systemic infection (e.g., growing organisms detected in blood cultures within 1-2 days after sample collection) in a very sick patient already admitted to a hospital because they have relatively low toxicity. However, because anti-fungal drugs can have more deleterious side effects, physicians often will not administer fungicides until the diagnosis is confirmed by a laboratory blood culture, and this is particularly true in children or in adults without any existing evidence of immune compromise. Moreover, although wide spectrum antibiotics can suppress many infections, it is generally more effective to administer a drug that preferentially and more effectively targets the specific type of pathogen, and hence, administration of optimal antibiotic therapy is commonly delayed for days until the results of laboratory blood culture pathogen typing and drug sensitivity assays can be obtained.

Even though blood culture assays remain the gold standard for clinical pathogen identification and typing, they can not identify the specific pathogen type for days (usually at least 3 to 7 days after the sample is collected and cultured on agar plates), which is an extremely long time for a patient with systemic infection or sepsis because their condition can degrade rapidly. It would therefore be extremely valuable to develop an inexpensive, robust, and simple-to-use POC diagnostic device that could be used in physician's private offices, by emergency medical technicians, and at homes and schools that could rapidly determine whether or not a patient with fever of unknown origin, chills or other generic clinical symptoms consistent with a blood borne infection indeed has this diagnosis, and hence requires immediate transfer to a hospital for a full workup.

Faster pathogen diagnostic methods based on genetic polymerase chain reaction (PCR), mass spectrometry or immunoassays are currently being explored in research laboratories, and some have been approved for use outside the United States. However, these complex assays are expensive, complex technically and difficult to implement in physician's offices or clinical labs. They also can be too sensitive to reagents commonly used in laboratory assays that are produced in genetically engineered microorganisms (e.g., in the case of PCR), and to natural microbial inhabitants of our body, especially in complex biological samples, such as whole blood that is sampled by passing a needle through the skin which has many normal microbial inhabitants 1. Thus, this method has been hampered by false positives, and it is limited by the fact that it can not detect whether the pathogens are living or dead, which further complicates the clinical prognosis and the development of a clinical action plan. Moreover, none of these methods have the simplicity or low cost that would make them useful for POC diagnostics that could be used in doctor's offices, ambulances, homes or schools, or for Global Health applications.

### SUMMARY OF THE INVENTION

The present invention is directed to a microfluidic device that facilitates the rapid separation and removal of target components for analysis and detection. The target components can be separated and removed from a source fluid flowing in a source microfluidic channel without removing or altering other components in the source fluid. The fluid can be a liquid or a gas. The target components can be any particulate, molecule or cellular material that is magnetic or can be bound to a magnetic particle introduced to the flowing fluid. Once separated from the fluid, the target components can be subject to any analysis and testing that can be used to detect the presence of any organic or inorganic material.

The method can include providing the microbeads having a coating adapted to bind with the target components and mixing the microbeads with the source fluid to be analyzed to enable one or more target component to become bound to one or more magnetic microbeads. The source fluid including the target components bound to the magnetic microbeads can be directed to flow through a microfluidic device that facilitates separation of the magnetic microbead from the fluid. The microfluidic device can include a microfluidic channel and capture chamber connected to the microchannel. A magnetic field gradient can be applied to the fluid in the microchannel causing the magnetic microbeads to migrate into the capture chamber. The target components can be analyzed and tested to detect the presence of any organic or inorganic material. The target components can be analyzed and tested in the capture chamber or the target components can be removed from the capture chamber for analysis and testing. The target components can be analyzed or tested in the capture chamber and then removed and subject to further testing.

The device can include a fluid inlet port, connected by one or more microchannels to a fluid outlet port to permit a flowing fluid to flow through the device. At least one of the microchannels can be connected to a capture chamber that can be adapted to collect and retain target components that migrate into the capture chamber. The device can also include a magnetic source that can produce a magnetic field gradient and apply the magnetic field gradient to the fluid flowing in the microchannel to cause the magnetic microbeads and the target components to migrate into the capture chamber. The target components can be analyzed and tested to detect the presence of any organic or inorganic material. The target components can be analyzed and tested in the capture chamber or the target components can be removed from the capture chamber for analysis and testing. The target components can be analyzed or tested in the capture chamber and then removed and subject to further testing.

The present invention can be used in the analysis and testing of both organic and inorganic fluids. In one embodiment of the invention, the source fluid can include a biofluid, such as human whole blood and the device can be used to rapidly detect pathogens. In an alternative embodiment of the invention, the source fluid includes water from a water supply or a liquid food source material and the device can be used to rapidly detect chemical and/or biological contamination.

### BRIEF DESCRIPTION OF THE DRAWINGS

The accompanying drawings illustrate an embodiment of the invention and depict the above-mentioned and other features of this invention and the manner of attaining them. In the drawings:

FIGS. 1A, 1B and 2 show a diagrammatic top and side view of a microfluidic device according to the invention.

FIG. 3 shows a diagrammatic view of a magnetic field gradient concentrator used separate magnetic microbeads according to the invention.

FIGS. 4A and 4B show a diagram of the magnetic field vectors and a graph of the magnetic flux density of the magnetic field concentrator shown in FIG. 3 according to the invention.

FIG. 5 shows a uniform geodesic magnetic bead array formed on a water drop surface according to the invention.

FIG. 6 a view of one C. albicans fungal cell captured from a 10 mL sample of human whole blood using a microfluidic device in accordance with the invention.

FIG. 7 is a graph showing a linear correlation between the concentration of fungal cells and the cell concentration detected by the microfluidic device according to the invention.

FIG. 8 shows images of C. albicans detection using phase contrast, calcoflour staining, secondary FITC conjugated antibodies and a merger of the three images.

FIG. 9 shows a schematic of detection of a pathogen in a biological sample according to one embodiment of the invention.

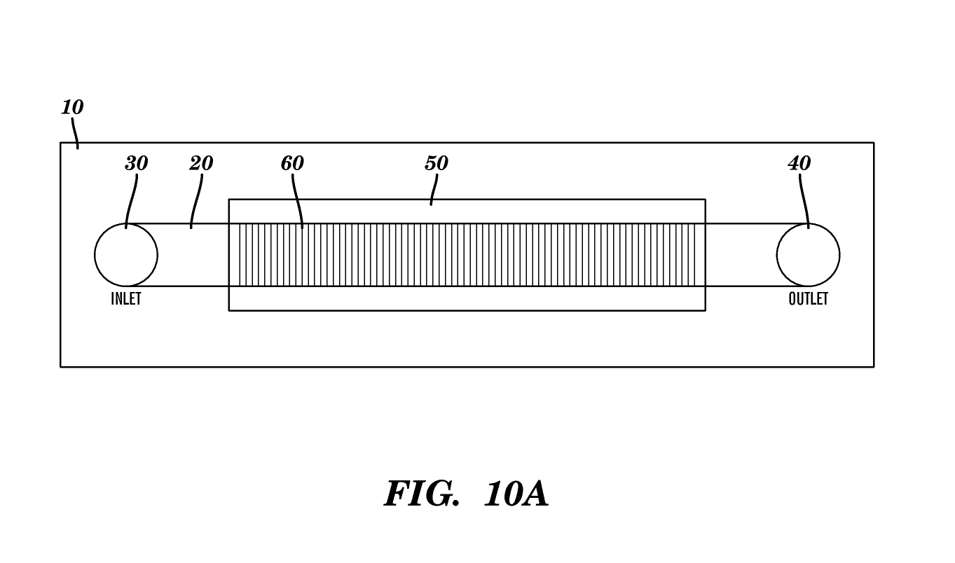

FIGS. 10A and 10B show a diagrammatic top and side view of a microfluidic device according to one embodiment of the invention. FIG. 10A, Schematic top view of the device. 480 um tall main channel represented in green, 80 um tall washboard on ceiling of capture chamber represented in red. FIG. 10B, Cross-section of the device showing the relationship of the magnet, flux concentrator, PDMS device and epifluorescence microscope. Small dots represent excess magnetic beads (100) while larger ovals represent pathogen (110) bound with magnetic beads.

FIGS. 11A-11E show the effect of the permanent magnet on accumulation of magnetic beads in the device. FIG. 11A, is a schematic showing magnetic particle tend to accumulate at lead edge of permanent magnet where ∇B has the greatest magnitude. FIG. 11B is a graph showing the magnetic field and force on superparamagnetic particles in the device channel. FIG. 11C is a schematic of the flux concentrator. FIG. 11D is a plot of the measured magnetic field 0.5 mm above the flux concentrator. Spikes at edges have been greatly reduced. FIG. 11E is a photograph showing the more uniform distribution of captured beads over the length of the capture chamber with a concentrator and magnet relative to a permanent magnet alone.

FIGS. 12A and 12B are line graphs showing analysis of C. albicans spiked blood sample. FIG. 12A shows results from control experiments where C. albicans were bound with beads before being spike into blood. Strong linear correlation between cfu/ml in sample and number of cells is seen in the device. FIG. 12B shows results from binding of C. albicans in blood. Data also shows a strong linear correlation between cfu/ml in sample and number of cell recovered. The epifluorescent images showed double staining of C. albicans with calcofluor (1 μM to 100 μM) and GFP (data not shown).

FIG. 13 is a brightfield image of magnetic beads in washboard at ceiling of capture chamber. The wash board gives a much better distribution of beads than an unpatterned capture chamber, improving visualization and quantification of captured pathogens.

FIG. 14 is a schematic of capturing and concentrating of micro- and nano-particles for optical resonator detection.

FIG. 15 is schematic showing specific detection of pathogens and biomarkers by detecting magnetic micro- or nano-particles that carry the analyte and that bind to recognition elements on the surface of the optical resonator.

FIG. 16 is a schematic showing the micro- and nano-particle detection principle, here for the example of a virion, with optical resonator.

FIG. 17 is a schematic showing label-free detection with a optical resonator biosensor.

FIG. 18A is a scanning electron microscope image of a toroidal resonator.

FIG. 18B is a fluorescene image of the toroidal resonator of FIG. 18A taken with epi-illumination of 200 nm-diameter polystyrene where particles have adsorbed and accumulated at the equatorial region of the optical resonator light orbit. The inset shows gray-scale values averaged for several linescans taken across the center of the toroid.

FIGS. 19A-19C show a diagrammatic illustration of the optical resonator detector component concept. FIG. 19A, a wavelength-tunable telecom laser delivers light (shown in red) through an optical fiber to a glass microsphere. FIG. 19B, at a specific resonance wavelength, the light couples to the microsphere, then no longer reaches the photodetector, a drop in the transmission intensity is recorded, the minimum of which corresponds to the resonance wavelength λ. FIG. 19C, ultra-sensitive detection of single influenza A virus particles has been accomplished by monitoring changes Δλ of the resonance wavelength, and detecting discrete steps in the wavelength as virus nanoparticles interact with the microsphere surface (Vollmer, et al., PNAS, 2008, 105:20701).

FIG. 20A is a graph showing resonance wavelength fluctuations Δλ/λ for radius a=250 nm PS particles interacting with a microsphere with radius R˜27 mm. WGM are excited at ˜63 nm nominal wavelength.

FIG. 20B is a graph showing simultaneously recorded fluctuations of resonance linewidth. The average Q-factor is measured ˜1×10⁶.

FIG. 21A is graph showing the shift signal for Influenza A virus particles. The data was acquired with a microscope cavity radius of R=39 μm, and a distributed feedback laser with a nominal wavelength of 763 nm.

FIG. 21B is an image showing fluorescently labeled influenza A virus particles. The particles bound to the microsphere cavity were imaged using a fluorescent microscope. We observed predominant binding of virions to the equator region of eh microsphere cavity, indicating a novel optical mechanism for nanoparticle trapping and accumulation.

### DETAILED DESCRIPTION OF THE INVENTION

The present invention is directed to a fluidic device that facilitates the rapid separation and removal of target components from a source fluid flowing in a microchannel without removing or altering other components in the source fluid. The fluid can be a liquid or a gas. The target components can be any particulate, molecule or cellular material that is magnetic or can be bound to a magnetic particle introduced to the fluid. The target components can be collected in a capture chamber and subject to analysis for detection of pathogens and/or contaminants. The target components can be analyzed and/or tested in the capture chamber or removed from the capture chamber for analysis and/or testing. The target components can be analyzed and/or tested, for example, using well known detection techniques to detect pathogens and/or contaminants.

In order to facilitate a better understanding of the invention, an illustrative example of an embodiment of the invention is described in the context of detecting pathogens in biological fluids, such as human whole blood. However, as one of ordinary skill will appreciate, the present invention can be embodied in systems used in other contexts.

In one embodiment, the present invention is directed to a microfluidic device and method that facilitates the rapid separation and of pathogens from a source biological fluid without removing or altering other components in the fluid. The target components can be any pathogen (particulate, molecule or cellular material) that is magnetic or can be bound to a magnetic particle introduced to the flowing fluid. The target components can be separated and collected in a capture chamber where they can be analyzed for rapid detection of pathogens. The target components can be analyzed and/or tested using well known analysis and testing techniques, such as immunostaining, culturing, polymerase chain reaction (PCR), mass spectrometry, optical resonance sensing and antibiotic sensitivity testing can be used to detect pathogens. Because target components are either magnet or bound to magnetic microbeads, magnetic field gradients can be used to further manipulate the target components to facilitate rapid detection.

In one embodiment, the present invention is directed to a low-cost, easy-to-operate microfluidic device and system for rapid pathogen detection. The device, according to one embodiment can identify clinically-relevant levels of Candida albicans (C. albicans) fungi (1-100 cell/mL) in whole human blood within minutes. The diagnostic system utilizes immunomagnetic beads and magnetic field gradients applied within localized regions of a microfluidic device to specifically bind, concentrate and immobilize the blood-borne pathogens. Fungal cells can be readily identified within the device by inspection with a common light microscope, with a fluorescent microscope after staining with calcofluor (1 μM to 100 μM), which is specific to fungal cell walls or using other in-chip detection methods. The present invention can be used to identify extremely low concentrations (<1 pathogen cell/mL) of C. albicans fungal cells in 10 mL of whole human blood within 45 min after sample collection with no requirement for sample pre-processing. The present invention can also be used to identify clinically-relevant levels of pathogens (0.5 to 100 colony forming units (cfu)/ml) in whole blood within minutes.

One of the challenges in this approach is that magnetically-isolated beads and bound pathogens can become densely packed within the magnetic collection chamber of the device, and thus, the optical opacity of the beads can visually obscure the rare pathogens during visual inspection. To avoid this problem, the invention can take advantage of magnetic properties of the magnet microbeads bound to the target pathogen and manipulate the beads using magnetic fields to spread out the microbeads prior to optical analysis, or alternative detection methods that require no label, such as optical resonance imaging, may be utilized for this purpose The detection limit of this diagnostic system also can be further increased by manipulating magnetic field distributions using specific stationary magnet configurations so as to uniformly spread out the isolated magnetic microbeads and bound target pathogens, without requiring any additional energy source.

The detection system according to the present invention can include magnetic microbeads, a mixing chamber or device for mixing the magnetic microbeads with the fluid, a microfluidic device having a capture chamber for separating the target components bound to the magnetic microbeads, a magnetic source providing a magnetic field gradient that can be applied to the fluid flowing through the microfluidic device to cause the magnetically bound target components to migrate into the capture chamber, a system for spreading the magnetic microbeads as necessary to facilitate detection and a pathogen detection component or system.

The method according to the invention can include selecting the microbeads having one or more coatings adapted to bind with one or more target components and mixing the microbeads with the source fluid to be analyzed to enable one or more target component to become bound to one or more magnetic microbeads. The source fluid including the target components bound to the magnetic microbeads can be directed to flow through a microfluidic device that facilitates separation of the magnetically bound target components from the fluid. The microfluidic device can include a microfluidic channel and a capture chamber connected to the microchannel. A magnetic field gradient can be applied to the fluid in the microchannel causing the magnetically bound target components to migrate into the capture chamber. The target components can be analyzed and tested to detect the presence of any organic or inorganic material, pathogen or contaminant. The target components can be analyzed and tested in the capture chamber or the target components can be removed from the capture chamber for analysis and testing. The target components can be analyzed or tested in the capture chamber and then removed and subject to further testing. A magnet field gradient can be used to separate or arrange the magnetically bound target components into an array to facilitate analysis and detection.

In accordance with one embodiment of the invention, a sample of blood or other biological fluid can be drawn from a patient into a syringe. After the needle is removed the, the syringe can be connected to a similar connection to allow the biological fluid to be injected into the a microfluidic device according to one embodiment of the invention. In biological fluid, for example human whole blood, can be injected into a reservoir of the microfluidic device that provides optimal mixing of the blood with the magnetic microbeads and causes the mixture of blood and magnetic microbeads to flow through the microchannels of the device. The capture chamber can rapidly collect the pathogens which can be analyzed using any known methods or techniques. For example, a stain or dye can be injected into the capture chamber to facilitate identification of pathogens using light microscopy.

### Microdevice I

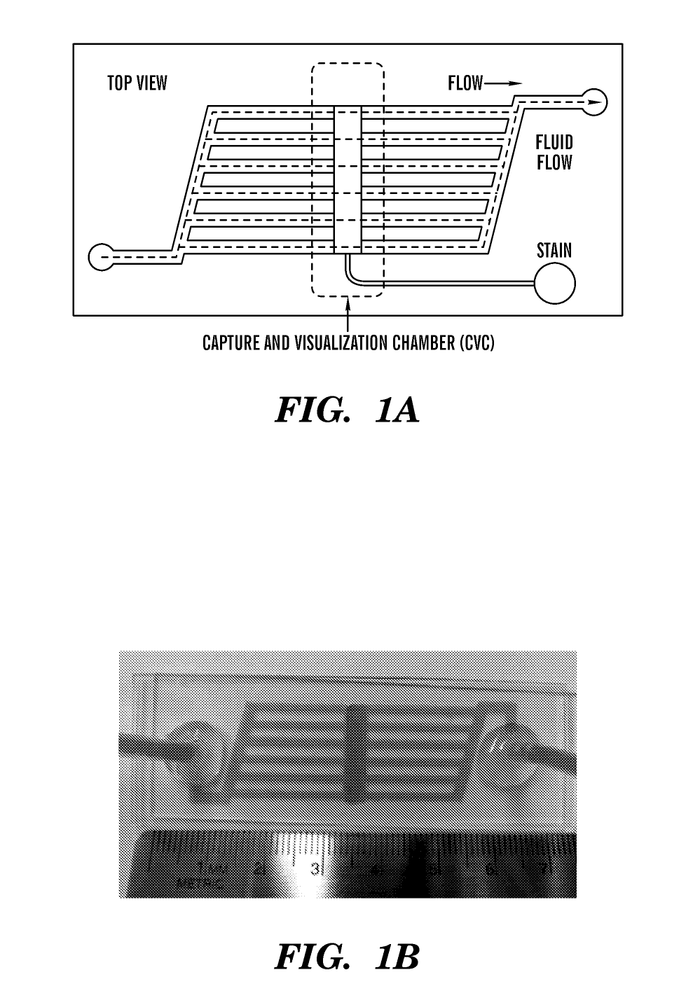

FIGS. 1A, 1B, and 2 show a microfluidic device according to one embodiment of the present invention. The microfluidic device can include one or more microchannels extending between an inlet port and an outlet port. The fluid, such as blood, can be injected into the inlet port and caused to flow through one or more of the microchannels to the outlet port. The microfluidic device can also include a capture chamber or capture and visualization chamber connected to one or more of the microchannels. The example shown in FIGS. 1A and 1B includes six microchannels and one capture and visualization chamber extending transverse to the microchannels, however devices according to the invention can include fewer or more microchannels. FIG. 2 shows the side view of the microfluidic device according to one embodiment of the invention. The capture and visualization chamber is connected to the microchannel and the microchannel, in this embodiment, extends through, or adjacent to, the capture and visualization chamber. Magnets can be placed above the capture and visualization chamber, providing a magnetic field gradient that extends into the fluid flowing in the microchannel. As shown in FIG. 2, the magnetic field gradient causes the magnetically bound target components (pathogens) to migrate into the capture and visualization chamber. As shown in FIG. 1A and 1B, the device includes a micromolded reservoir with a channel connected to the capture and visualization chamber. Dyes, stains and other analysis or testing components can be stored in the reservoir and pumped or injected into the capture and visualization chamber to facilitate detection.

### Microdevice II

In the device described above, magnetically-isolated beads and bound pathogens are densely packed within the magnetic collection chamber of the device, and thus, the optical opacity of the beads can visually obscure the rare pathogens during visual inspection. The structure of the device and the shape of the magnetic field can be optimized to prevent this dense packing of magnetic particles from occurring so that the captured pathogens can be clearly viewed during the identification step.

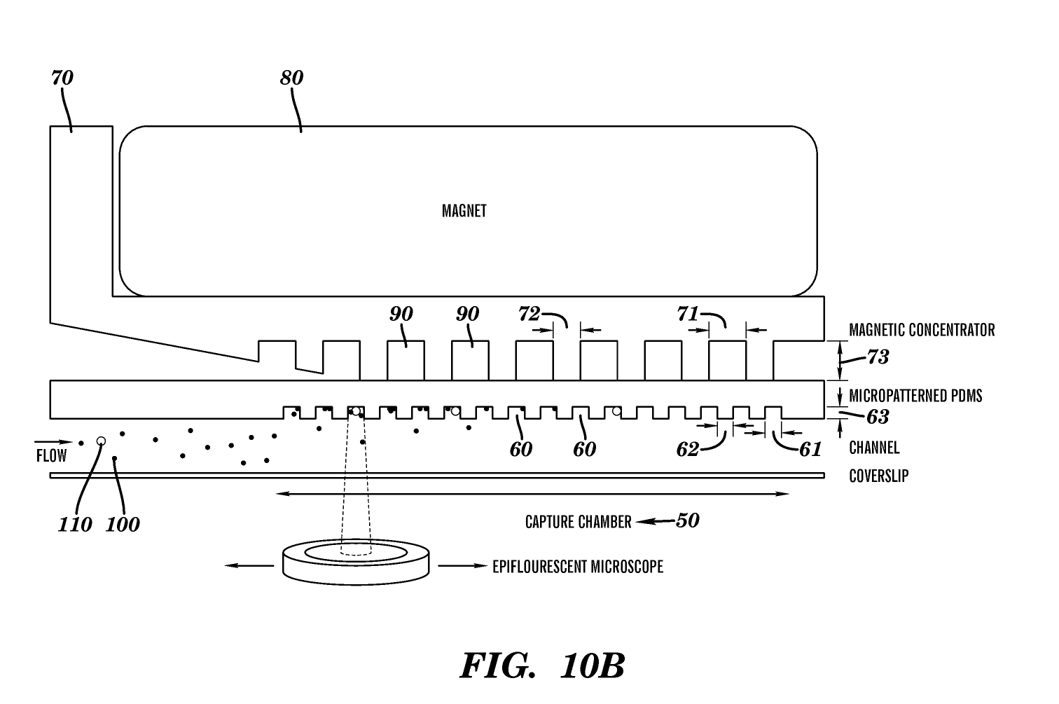

Accordingly, FIGS. 10A and 10B show a microfluidic device (10) according to another embodiment of the present invention. The microfluidic device can include one or more microchannels (20) extending between an inlet port (30) and an outlet port (40). The source fluid can be injected into the inlet port and caused to flow through one or more of the microchannels to the outlet port. The device can include one or more capture chambers or capture and visualization chambers (50). The capture and visualization chamber can be a region of the channel that is engineered with microfeatures, e.g., grooves or microchannels (60) to enhance retention of magnetically-separated target components (e.g., pathogens). However, other configurations (e.g. saw-tooth shaped steps, ridges and projections) can be used to similarly increase capture of bead-bound pathogen cells, or other bound particulates.

Dimension of the microchannel (20) can be chosen based on the specific application of the device. Accordingly, width of the microchannel (20) can range from about 0.1 mm to about 10 mm. In some embodiments, width of the microchannel (20) is from about 0.5 mm to about 5 mm. In some embodiments, width of the microchannel (20) is from about 1 mm to about 4 mm. In some embodiments, width of the microchannel is about 2.5 mm.

Depth or height of the microchannel (20) can also be chosen based on the specific application of the device. Accordingly, depth of the microchannel (20) can range from about 50 μm to about 2000 μm. In some embodiments, depth of the microchannel is from about 100 μm to about 1000 μm. In some embodiments, depth of the microchannel is from about 250 μm to about 750 μm. In some embodiments, depth of the microchannel is about 560 μm.

In one embodiment, the microchannel (20) comprises a plurality of grooves or microchannels (60) extending transverse to the channel in the capture and visualization chamber. The grooves can be of same dimension or of different dimensions, and the dimensions of the grooves can be optimized for the particular application of the device. The spacing between the grooves can be same between all grooves or different between different grooves. Accordingly, the grooves can form a regular or irregular pattern in the capture and visualization region. For example, the grooves can form a regular washboard-like feature in the channel.

Dimensions of the grooves (60) are such that as to retain one or more magnetic-beads in the groove. In other words, dimension of the groove are such that flow of a magnetic bead in the groove will be impeded. Thus, width (61) of the groove is larger than the diameter of the magnetic-beads to be used in the device. According, in some embodiments, width of the groove is from about 0.1 μm to about 1000 μm. In some embodiments, width of the groove is from about 50 μm to about 250 μm. In some embodiments, width of the groove is from about 75 μm to about 150 μm. In one embodiment, width of the groove is about 100 μm.

As described above, the grooves can from a regular pattern in the microchannel (20). Accordingly, the spacing (62) between the grooves can range from about 0.1 μm to about 1000 μm. In some embodiments, spacing between the groove is from about 50 μm to about 500 μm, from about 75 μm to about 300 μm, from about 100 μto about 250 μm. In one embodiment, spacing between the grooves is about 200 μm.

Similarly, depth or height (63) of the grooves (60) can range from about 0.1 μm to about 500 μm. In some embodiments, depth of the groove is from about 25 μm to about 250 μm, from about 50 μm to about 200 μm, from about 75 μm to about 150 μm. In one embodiment, depth of the groove is about 80 μm.

The capture or the capture and visualization chamber (50) can comprise all of the microchannel (20) or part of the microchannel (20).

The example shown in FIG. 10A includes one channel comprising one capture and visualization chamber, however devices according to the invention can one or more channels and/or capture and visualization chamber. For example, the device can comprise 1, 2, 3, 4, 5, 6, 7, 8, 9, 10 or more channels and/or capture and visualization chambers.

FIG. 10B shows the side view of the microfluidic device according to one embodiment of the invention. A magnetic concentrator (70), also referred to as a magnetic flux concentrator herein, can be placed above the capture and visualization chamber. Placement of a magnet (80) on the magnetic concentrator can then provide a more uniform magnetic field gradient, which extends into the fluid in the channel, along the length of the capture and visualization chamber. The surface of the magnetic concentrator, which faces the channel, can have a plurality of grooves (90).

Magnetically tagged pathogens (110) can be pulled to the surface of the capture chamber by the magnetic field gradient where they settle into the washboard grooves (60), which shields them from the fluid flow and greatly reduces the fluidic drag they experience, preventing them from being swept downstream (FIG. 10B). The magnetic concentrator can reinforce this by locally angling the magnetic field so that the force on the beads directly opposes the fluidic drag as well.

The pattern of grooves in the magnetic concentrator can match the pattern of grooves in the channel. Accordingly, when the magnetic concentrator is placed above the capture and visualization region, the grooves in the magnetic concentrator can align with or be partially or completely offset from the grooves in the device channel.

The width (71) and the spacing (72) of the grooves (90) in the magnetic concentrator can match the width and spacing of the grooves in the device channel. For example, width (71) of a groove (90) of the magnetic concentrator can be of the same or an integer multiple of the width (61) of a groove in the device channel and the spacing (72) between the grooves in the magnetic concentrator can be the same or an integer multiple of the spacing (62) between the grooves in the device channel. In another example, width (71) of a groove of the magnetic concentrator can be of the same or an integer multiple of the spacing (62) between the grooves in the device channel and the spacing (72) between the grooves in the magnetic concentrator can be the same or an integer multiple of the width (61) of a groove in the device channel.

Alternatively, the width (71) and the spacing (72) of the grooves in the magnetic concentrator can be designed as not to match the width and spacing of the grooves in the device channel. For example, the spacing between the magnetic concentrator grooves is larger than the width of a groove in the device channel. In another example, the spacing between the magnetic concentrator grooves is smaller than the width of a groove in the device channel.

As described above, width and spacing of the groves (90) on the surface of the magnetic concentrator can be similar to those of the grooves (60) in the microchannel. Accordingly, the spacing (72) between the grooves can range from about 10 μm to about 1000 μm. In some embodiments, spacing between the grooves is from about 50 μm to about 500 μm, from about 75 μm to about 300 μm, from about 100 μm to about 250 μm. In one embodiment, spacing between the grooves is about 400 μm.

The width (71) of the groove (90) can range from about 10 μm to about 1000 μm. In some embodiments, width of the groove is from about 50 μm to about 250 μm. In some embodiments, width of the groove is from about 75 μm to about 150 μm. In one embodiment, width of the groove is about 100 μm. In one embodiment, width of the groove is about 400 μm.

Depth or height (73) of the grooves (90) can range from about 10 μm to about 2000 μm. In some embodiments, depth of the groove is from about 150 μm to about 1500 μm, from about 250 μm to about 1000 μm, from about 350 μm to about 750 μm. In one embodiment, depth of the groove (90) is about 400 μm.

The microdevice can include one or more micromolded reservoirs with a channel connected to the capture and visualization chamber. Dyes, stains and other analysis or testing components can be stored in the reservoir and pumped or injected into the capture and visualization chamber to facilitate detection.

### Source Fluid Flow Rate

The skilled artisan is well aware that the flow of the source fluid through a microdevice is dependent on various factors including, but not limited to, dimensions of the microchannels, viscosity of the source fluid, target component to be separated, the detection and method employed. Accordingly, the source fluid can flow through the microdevice microchannel at a rate of about 1 ml/hr to about 100 L/hr. In some embodiments, the source fluid can flow through the microdevice microchannel at a rate of about 1 ml/hr to about 100 ml/hr, about 5 ml/hr to about 50 ml/hr, from about 7.5 ml/hr to about 25 ml/hr, or about 10 ml/hr to about 20 ml/hr. In one embodiments, the source fluid flows at a rate of about 15 ml/hr.

### Magnetic Beads

The magnetic microbeads can be, for example, super-paramagnetic microbeads (0.1 to 10 um diameter) that are coated using conventional techniques with antibodies or other molecules (e.g., aptamers, surface receptor ligands, etc.) that specifically bind to the surface of pathogenic cells in complex fluids, such as whole blood.

The magnetic microbead can be of any shape, including but not limited to, spherical, rod, elliptical, cylindrical, disc, and the like. In some embodiments, magnetic microbeads having a true spherical shape and defined surface chemistry are used to minimize chemical agglutination and non-specific binding. As used herein, the term “magnetic bead” refers to a nano- or micro-scale particle that is attracted or repelled by a magnetic field gradient or has a non-zero magnetic susceptibility. The term “magnetic microbead” also includes magnetic microbeads that have been conjugated with affinity molecules. The magnetic microbeads can be paramagnetic or super-paramagnetic microbeads. In some embodiments, the magnetic microbeads are super-paramagnetic. Magnetic beads are also referred to as beads herein.

In some embodiments, magnetic microbeads having a polymer shell are used to protect the target component from exposure to iron. For example, polymer coated magnetic microbeads can be used to protect target cells from exposure to iron. In some embodiments, the magnetic microbeads or beads can be selected to be compatible with the fluids being used, so as not to cause undesirable changes to the source fluid. For example, for biofluids, the magnetic microbeads can made from well know biocompatible materials.

The magnetic microbeads can range in size from 1 nm to 1 mm. Preferably magnetic microbeads are about 250 nm to about 250 μm in size. In some embodiments, magnetic particle is 0.1 μm to 50 μm in size. In some embodiments, magnetic particle is 0.1 μm to 10 μm in size. In some embodiments, the magnetic particle is a magnetic nano-particle or magnetic microparticle. Magnetic nanoparticles are a class of nanoparticle which can be manipulated using magnetic field. Such particles commonly consist of magnetic elements such as iron, nickel and cobalt and their chemical compounds. Magnetic nano-particles are well known and methods for their preparation have been described in the are art, for example in U.S. Pat. Nos. 6,878,445; 5,543,158; 5,578,325; 6,676,729; 6,045,925 and 7,462,446, and U.S. Pat. Pub. Nos. 2005/0025971; 2005/0200438; 2005/0201941; 2005/0271745; 2006/0228551; 2006/0233712; 2007/01666232 and 2007/0264199, contents of all of which are herein incorporated by reference in their entirety.

Magnetic microbeads are easily and widely available commercially, with or without functional groups capable of binding to affinity molecules. Suitable superparamagnetic microbeads are commercially available such as from Dynal Inc. of Lake Success, N.Y.; PerSeptive Diagnostics, Inc. of Cambridge, Mass.; Invitrogen Corp. of Carlsbad, Calif.; Cortex Biochem Inc. of San Leandro, Calif.; and Bangs Laboratories of Fishers, Ind. In some embodiments, magnetic microbeads are Dynal Magnetic beads such as MyOne Dynabeads. In some embodiments, the magnetic microbeads are microbeads coated with MBL (mannose binding lectin) as described in U.S. Prov. App. No. 61/296,222, filed Jan. 19, 2010, content of which is incorporated herein in its entirety. These MBL coated magnetic microbeads are also referred to as engineered Opsonin. To clarify, by “MBL coated magnetic microbead” is meant a magnetic microbead that is coated with a carbohydrate recognition domain of an Opsonin, i.e, at least one carbohydrate recognition domain of an Opsonin is present on the surface of microbead. The carbohydrate recognition domain can be linked to the surface of the microbead either directly or through a linker. The linker can be a peptide linker, for example.

### Magnetic Particle—Target Component Binding

The degree of magnetic particle binding to a target component is such that the bound target component will move when a magnetic field is applied. It is to be understood that binding of magnetic particle with the target component is mediated through affinity molecules, i.e., the affinity molecule on the surface of the magnetic particle that binds to the target component. Binding of magnetic microbeads to target components can be determined using methods or assays known to one of skill in the art, such as ligand binding kinetic assays and saturation assays. For example, binding kinetics of a target component and the magnetic particle can be examined under batch conditions to optimize the degree of binding. In another example, the amount of magnetic microbeads needed to bind a target component can be ascertained by varying the ratio of magnetic microbeads to target component under batch conditions. Without wishing to be bound by theory, the binding efficiency can follow any kinetic relationship, such as a first-order relationship. In some embodiments, binding efficiency follows a Langmuir adsorption model.

The separation efficiency of a microfluidic device described herein can be determined using methods known in the art and easily adaptable for microfluidic devices. For example, magnetic particle conjugated with an affinity molecule and the target component are pre-incubated in the appropriate medium to allow maximum binding before resuspending in a source fluid such as a biological fluid. The effects of varying electromagnet current on separation efficiency can be analyzed using, for example, target component—magnetic particle complexes suspended in PBS. To test how the viscosity of the collection fluid affected its hydrodynamic interaction with a biological fluid, such as blood, medical grade dextran (40 kDa, Sigma) can be used to vary the viscosity. For example, dextran can be dissolved in PBS at 5, 10 and 20% to produce solutions with viscosities of 2, 3, 11 centipoise at room temperature. Samples can be collected from bottom-inlet, top-outlet, and bottom-outlet channels and analyzed by flow cytometry to assess the separation efficiency of magnetic microbeads and particle bound target components. Efficiency can be calculated as: Efficiency=1−X bottom-out/X bottom-in. Source fluid loss can be quantified using an appropriate marker in the source fluid. For example, blood loss can be quantified by measuring the OD600 of red blood cells (Loss=OD top-out/OD bottom-out).

The optimal time for binding of magnetic microbeads to target component can vary depending on the particulars of the device or methods being employed. The optimal mixing and/or incubation time for binding of magnetic microbeads to a target component can be determined using kinetic assays well known to one of skill in the art. For example, kinetic assays can be performed under conditions that mimic the particulars of the device or methods to be employed, such as volumes, concentrations, how and where the mixing is to be performed, and the like. The rate of binding of magnetic microbeads to target components can be increased by carrying out mixing within separate microfluidic mixing channels.

### Magnetic Field Gradient

The magnetic gradient can be generated by a permanent magnet or by an electromagnetic signal generator. The electromagnetic signal generator can include an electromagnet or electrically-polarizable element, or at least one permanent magnet. The magnetic gradient can be produced at least in part according to a pre-programmed pattern. The magnetic gradient can have a defined magnetic field strength and/or spatial orientation. In some embodiments, the magnetic gradient has a defined magnetic field strength. As used herein, the term “magnetic field” refers to magnetic influences which create a local magnetic flux that flows through a composition and can refer to field amplitude, squared-amplitude, or time-averaged squared-amplitude. It is to be understood that magnetic field can be a direct-current (DC) magnetic field or alternating-current (AC) magnetic field. Magnetic field strength can range from about 0.001 Tesla to about 1 Tesla. In some embodiments, magnetic field strength is in the range from about 0.01 Tesla to about 1 Tesla. In some other embodiments, magnetic field strength is in the range from about 0.1 Tesla to about 1 Tesla.

Binding/Affinity Molecules

The surfaces of the magnetic microbeads are functionalized to include binding molecules that bind selectively with the target component. These binding molecules are also referred to as affinity molecules herein. The binding molecule can be bound covalently or non-covalently (e.g. adsorption of molecule onto surface of the particle) to each magnetic particle. The binding molecule can be selected such that it can bind to any part of the target component that is accessible. For example, the binding molecule can be selected to bind to any antigen of a pathogen that is accessible on the surface, e.g., a surface antigen.

As used herein, the term “binding molecule” or “affinity molecule” refers to any molecule that is capable of specifically binding a target component. Representative examples of affinity molecules include, but are not limited to, antibodies, antigens, lectins, proteins, peptides, nucleic acids (DNA, RNA, PNA and nucleic acids that are mixtures thereof or that include nucleotide derivatives or analogs); receptor molecules, such as the insulin receptor; ligands for receptors (e.g., insulin for the insulin receptor); carbohydrates; and biological, chemical or other molecules that have affinity for another molecule, such as biotin and avidin. The binding molecules need not comprise an entire naturally occurring molecule but may consist of only a portion, fragment or subunit of a naturally or non-naturally occurring molecule, as for example the Fab fragment of an antibody. The binding molecule may further comprise a marker that can be detected.

Nucleic acid based binding molecules include aptamers. As used herein, the term “aptamer” means a single-stranded, partially single-stranded, partially double-stranded or double-stranded nucleotide sequence capable of specifically recognizing a selected non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

In some embodiments of the aspects described herein, the binding molecules specific are polyclonal and/or monoclonal antibodies and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. Accordingly, as used herein, the term “antibody” refers to an intact immunoglobulin or to a monoclonal or polyclonal antigen-binding fragment with the Fc (crystallizable fragment) region or FcRn binding fragment of the Fc region. Antigen-binding fragments may be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)]. Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

In some embodiments, the binding molecule binds with a cell-surface marker or cell-surface molecule. In some further embodiments, the binding molecule binds with a cell-surface marker but does not cause initiation of downstream signaling event mediated by that cell-surface marker. Binding molecules specific for cell-surface molecules include, but are not limited to, antibodies or fragments thereof, natural or recombinant ligands, small molecules, nucleic acids and analogues thereof, intrabodies, aptamers, lectins, and other proteins or peptides.

As used herein, a “cell-surface marker” refers to any molecule that is present on the outer surface of a cell. Some molecules that are normally not found on the cell-surface can be engineered by recombinant techniques to be expressed on the surface of a cell. Many naturally occurring cell-surface markers present on mammalian cells are termed “CD” or “cluster of differentiation” molecules. Cell-surface markers often provide antigenic determinants to which antibodies can bind to.

Accordingly, as defined herein, a “binding molecule specific for a cell-surface marker” refers to any molecule that can selectively react with or bind to that cell-surface marker, but has little or no detectable reactivity to another cell-surface marker or antigen. Without wishing to be bound by theory, affinity molecules specific for cell-surface markers generally recognize unique structural features of the markers. In some embodiments of the aspects described herein, the preferred affinity molecules specific for cell-surface markers are polyclonal and/or monoclonal antibodies and antigen-binding derivatives or fragments thereof.

The binding molecule can be conjugated to the magnetic particle using any of a variety of methods known to those of skill in the art. The affinity molecule can be coupled or conjugated to the magnetic microbeads covalently or non-covalently. The covalent linkage between the affinity molecule and the magnetic particle can be mediated by a linker. The non-covalent linkage between the affinity molecule and the magnetic particle can be based on ionic interactions, van der Waals interactions, dipole-dipole interactions, hydrogen bonds, electrostatic interactions, and/or shape recognition interactions.

As used herein, the term “linker” means an organic moiety that connects two parts of a compound. Linkers typically comprise a direct bond or an atom such as oxygen or sulfur, a unit such as NH, C(O), C(O)O, OC(O)O, C(O)NH, OC(O)NH, NHC(O)NH, SO, SO₂, SO₂NH or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, C(O).

In some embodiments, the binding molecule is coupled to the magnetic particle by use of an affinity binding pair. The term “affinity binding pair” or “binding pair” refers to first and second molecules that specifically bind to each other. One member of the binding pair is conjugated with the magnetic particle while the second member is conjugated with the affinity molecule. As used herein, the term “specific binding” refers to binding of the first member of the binding pair to the second member of the binding pair with greater affinity and specificity than to other molecules.

Exemplary binding pairs include any haptenic or antigenic compound in combination with a corresponding antibody or binding portion or fragment thereof (e.g., digoxigenin and anti-digoxigenin; mouse immunoglobulin and goat antimouse immunoglobulin) and nonimmunological binding pairs (e.g., biotin-avidin, biotin-streptavidin, hormone [e.g., thyroxine and cortisol-hormone binding protein, receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleotide pairs capable of forming nucleic acid duplexes), and the like. The binding pair can also include a first molecule which is negatively charged and a second molecule which is positively charged.

In some cases, the target component comprises one member of an affinity binding pair. In such cases, the second member of the binding pair can be conjugated to a magnetic particle as an affinity molecule.

In some embodiments, the target component is first conjugated to one member of an affinity binding pair, and the second member of the affinity binding pair is conjugated to the magnetic particle.

In some embodiments, the magnetic particle is functionalized with two or more different affinity molecules. The two or more different affinity molecules can target the same target component or different target components. For example, a magnetic particle can be functionalized with antibodies and lectins to simultaneously target multiple surface antigens or cell-surface markers. In another example, a magnetic particle can be functionalized with antibodies that target surface antigens or cell-surface markers on different cells, or with lectins, such as mannose-binding lectin, that recognizes surface markers on a wide variety of pathogens.

In some embodiments, the binding/affinity molecule is a ligand that binds to a receptor on the surface of that target cell. Such a ligand can be a naturally occurring molecule, a fragment thereof or a synthetic molecule or fragment thereof. In some embodiments, the ligand is non-natural molecule selected for binding with a target cell. High throughput methods for selecting non-natural cell binding ligands are known in the art and easily available to one of skill in the art. See for example, Anderson, et al., Biomaterial microarrays: rapid, microscale screening of polymer-cell interaction. Biomaterials (2005) 26:4892-4897; Anderson, et al., Nanoliter-scale synthesis of arrayed biomaterials and application to human embryonic stem cells. Nature Biotechnology (2004) 22:863-866; Orner, et al., Arrays for the combinatorial exploration of cell adhesion. Journal of the American Chemical Society (2004) 126:10808-10809; Falsey, et al., Peptide and small molecule microarray for high throughput cell adhesion and functional assays. Bioconjugate Chemistry (2001) 12:346-353; Liu, et al., Biomacromolecules (2001) 2(2): 362-368; and Taurniare, et al., Chem. Comm. (2006): 2118-2120.

In some embodiments, the binding molecule and/or the magnetic microbeads can be conjugated with a label, such as a fluorescent label or a biotin label. When conjugated with a label, the binding molecule and the magnetic particle are referred to as “labeled binding molecule” and “labeled magnetic microbeads” respectively. In some embodiments, the binding molecule and the magnetic microbeads are both independently conjugated with a label, such as a fluorescent label or a biotin label. Without wishing to be bound by theory, such labeling allows one to easily track the efficiency and/or effectiveness of methods to selectively bind the target component in a source fluid. For example, a multi-fluorescence labeling can be used to distinguish between free magnetic microbeads, free target components and magnetic particle—target component complexes.

As used herein, the term “label” refers to a composition capable of producing a detectable signal indicative of the presence of a target. Suitable labels include fluorescent molecules, radioisotopes, nucleotide chromophores, enzymes, substrates, chemiluminescent moieties, magnetic microbeads, bioluminescent moieties, and the like. As such, a label is any composition detectable by spectroscopic, photochemical, biochemical, immunochemical, electrical, optical or chemical means needed for the methods and devices described herein. For example, binding molecules and/or magnetic microbeads can also be labeled with a detectable tag, such as c-Myc, HA, VSV-G, HSV, FLAG, V5, or HIS, which can be detected using an antibody specific to the label, for example, an anti-c-Myc antibody.

Exemplary fluorescent labels include, but are not limited to, Calcofluor (Calcofluor-white), Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

As used herein, a “labeling molecule” refers to a molecule that comprises a label and can bind with a target component. Accordingly, labeling molecules include, binding molecules described herein that comprise one or more labels as that term is described herein.

The mixing chamber or device can include a reservoir and/or a low-shear mixer or magnetic agitator, to mix the magnetic microbeads with fluid, such as whole human blood or other complex biological fluids (e.g., cerebral spinal fluid, sputum, urine, etc.).

The magnetic source can be one or more rare earth magnets positioned adjacent to the microchannel to generate the magnetic field gradients that are used to magnetically pull the microbead-bound target components (e.g., pathogens) out from the flowing fluid, e.g. blood. The magnetic source can also be formed from one or more electro-magnets positioned adjacent to the microchannel. An electromagnetic controller can be used to control and adjust the magnetic field gradients and control the migration, separation and orientation of the magnetically bound target components (e.g., pathogens).

### Device Fabrication

In accordance with one embodiment of the invention, a magnetically bound pathogen detection device can be fabricated by plasma bonding a single layer of micromolded PDMS to a standard microscope glass slide (1 inch×3 inch×1 mm; width×length×thickness) as shown in FIGS. 1 and 2. This micromolded PDMS can include a distributed network of six or more microfluidic flow channels (1.5 mm×2 cm×80 um; width×length×height) in parallel. The flow channels can be interconnected at their midpoint (length-wise) by a cavity or chamber (2 cm×4 mm×320 um; width×length×height), herein referred to as the Capture and Visualization Chamber (CVC). All channel features and the CVC can micromolded from a sticker-based mold fabricated using a cutter-plotter; however, conventional microfabrication techniques can also be utilized to produce these devices. Extremely strong rare earth neodymimum magnets (NdFeB) can be placed directly above the CVC to magnetically pull pathogens tagged with magnetic microbeads towards the ceiling or top surface of the CVC and away from the main fluid stream flow (e.g., blood sample) below. The larger cross-sectional area of the CVC can be provided to reduce the linear velocity of the fluid stream flow to further enhance magnetic separation of magnetically bound pathogens from the flowing blood, as well as to reduce shear forces acting on separated particles already resting on the surfaces of the CVC to minimize bead loss and suppress blood coagulation. As shown in FIG. 2, the downstream section of the CVC can be engineered with stepped microfeatures to further enhance retention of magnetically-separated target components (e.g., pathogens). However, other configurations (e.g. saw-tooth shaped steps, ridges and projections) can be used to similarly increase capture of bead-bound pathogen cells, or other bound particulates (e.g., inflammatory proteins, cytokines, auto-immune antibodies, etc.).

In accordance with another embodiment of the invention, a magnetic pathogen detection device can be fabricated by plasma bonding a single layer of micromolded polydimethylsiloxane (PDMS) (60×25×3 mm; width (w)×length (l)×height (h)) to a microscope glass slide (60×24×0.167 mm; width (w)×length (l)×height (h)) as shown in FIG. 10. This micromolded PDMS can include a single long channel (2.5 mm×4 cm×560 um; width (w)×length (l)×height (h)). The middle 20 mm of the channel length can include 100 um wide and 80 um deep grooves that repeat every 200 um, forming a regular washboard-like feature that comprises the ceiling of the capture chamber. Main channel feature can be micromolded from a sticker-based mold fabricated using a cutter-plotter and the washboard feature fabricated photolithographically using SU-8 molding. Conventional microfabrication techniques can also be utilized to produce these devices.

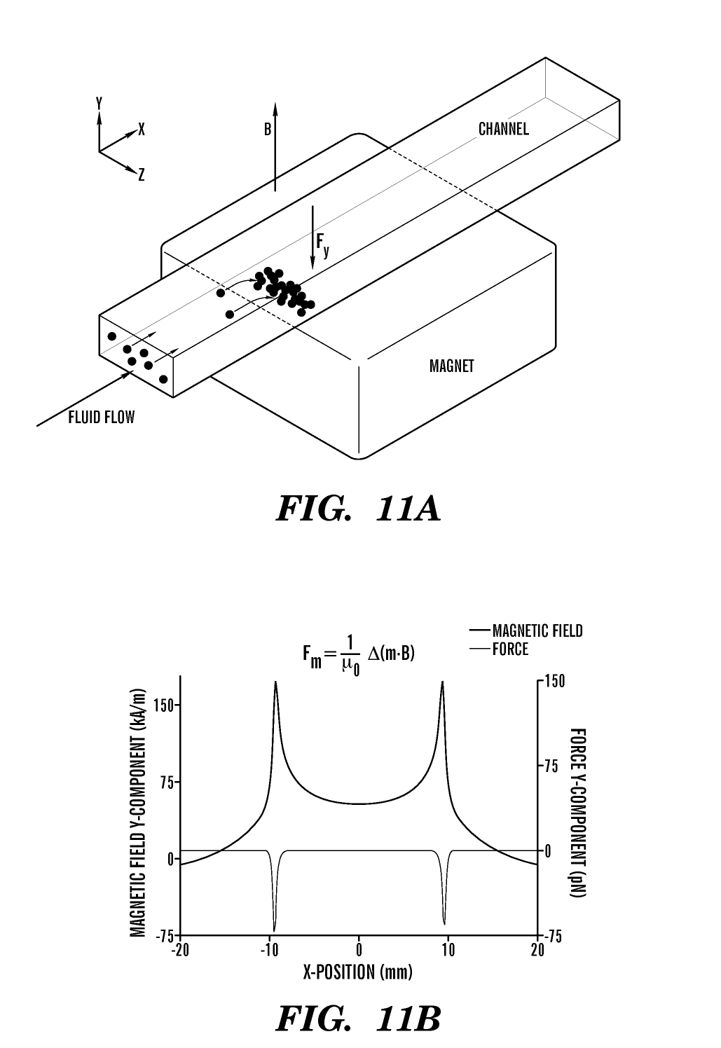

The magnetic concentrator can be micromachined from a high permeability magnetic material, in one embodiment EFI Alloy 79 (10 mm×25 mm×1.55 mm, width (w)×length (l)×height (h)) with the front 5 mm tapered to reduce the strength of magnetic field gradient followed by a repeating washboard of 400 um deep by 400 um long grooves that serve to angle and concentrate the magnetic field around them, giving a more uniform distribution of magnetic force on the particles in the capture chamber (FIGS. 10B and 11). The magnetic flux concentrator can be magnetized using a permanent neodynium magnet (NdFeB) (dimensions 0.75″×0.75″ 0.75″ width (w)×length (l)×height (h)). This combination creates a relatively more uniform magnetic field gradient along the length of the capture chamber than is possible with a permanent magnet alone (FIG. 11).

The microfluidic devices described herein can be fabricated from any biocompatible material. As used herein, the term “biocompatible material” refers to any polymeric material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood. Suitable biocompatible materials include derivatives and copolymers of a polyimides, poly(ethylene glycol), polyvinyl alcohol, polyethyleneimine, and polyvinylamine, polyacrylates, polyamides, polyesters, polycarbonates, and polystyrenes.

In some embodiments, the device is fabricated from a material selected from the group consisting of polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

In some embodiments, the device can be fabricated from materials that are compatible with the fluids used in the system. While the plastics described herein can be used with may fluids, some materials may break down when highly acidic or alkaline fluids are used and it is recognized that the removal of the target component from the source fluid can change the composition and characteristics of the source fluid. In these embodiments, other materials such as stainless steels, titanium, platinum, alloys, ceramics and glasses can be used. In addition, the channel(s) can be coated or treated to resist degradation or facilitate flow and operation. In some embodiments, it can be desirable to use different materials in the microchannel(s) and the capture chamber(s).

The magnetic concentrator can be made from any material having high magnetic permeability. Magnetic permeability (μ) is the measure of the ability of a material to support the formation of a magnetic filed within itself. In other words, it is the degree of magnetization that a material obtains in response to an applied magnetic field. Accordingly, the magnetic concentrator material can have a magnetic permeability of at least 10⁻⁵ H/m, or at least 10⁻⁴ H/m, or at least 10⁻³ H/m, or at least 10⁻² H/m, or at least 10⁻¹ H/m. In one embodiment, the magnetic concentrator is made from permalloy. The term “permalloy” generally refers to any of several alloys of nickel and iron having high magnetic permeability.

### Source Fluids

As used herein, the term “source fluid” refers to any flowable material that comprises the target component. Without wishing to be bound by theory, the source fluid can be liquid (e.g., aqueous or non-aqueous), supercritical fluid, gases, solutions, suspensions, and the like.

In some embodiments, the source fluid is a biological fluid. The terms “biological fluid” and “biofluid” are used interchangeably herein and refer to aqueous fluids of biological origin, including solutions, suspensions, dispersions, and gels, and thus may or may not contain undissolved particulate matter. Exemplary biological fluids include, but are not limited to, blood (including whole blood, plasma, cord blood and serum), lactation products (e.g., milk), amniotic fluids, sputum, saliva, urine, semen, cerebrospinal fluid, bronchial aspirate, perspiration, mucus, liquefied feces, synovial fluid, lymphatic fluid, tears, tracheal aspirate, and fractions thereof.

Another example of a group of biological fluids are cell culture fluids, including those obtained by culturing or fermentation, for example, of single- or multi-cell organisms, including prokaryotes (e.g., bacteria) and eukaryotes (e.g., animal cells, plant cells, yeasts, fungi), and including fractions thereof.

Yet another example of a group of biological fluids are cell lysate fluids including fractions thereof. For example, cells (such as red blood cells, white blood cells, cultured cells) may be harvested and lysed to obtain a cell lysate (e.g., a biological fluid), from which molecules of interest (e.g., hemoglobin, interferon, T-cell growth factor, interleukins) may be separated with the aid of the present invention.

Still another example of a group of biological fluids are culture media fluids including fractions thereof. For example, culture media comprising biological products (e.g., proteins secreted by cells cultured therein) may be collected and molecules of interest separated therefrom with the aid of the present invention.

In some embodiments, the source fluid is a non-biological fluid. As used herein, the term “non-biological fluid” refers to any aqueous, non-aqueous or gaseous sample that is not a biological fluid as the term is defined herein. Exemplary non-biological fluids include, but are not limited to, water, salt water, brine, organic solvents such as alcohols (e.g., methanol, ethanol, isopropyl alcohol, butanol etc.), saline solutions, sugar solutions, carbohydrate solutions, lipid solutions, nucleic acid solutions, hydrocarbons (e.g. liquid hydrocarbons), acids, gasolines, petroleum, liquefied samples (e.g., liquefied foods), gases (e.g., oxygen, CO2, air, nitrogen, or an inert gas), and mixtures thereof.

In some embodiments, the source fluid is a media or reagent solution used in a laboratory or clinical setting, such as for biomedical and molecular biology applications. As used herein, the term “media” refers to a medium for maintaining a tissue or cell population, or culturing a cell population (e.g. “culture media”) containing nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. The media can include media to which cells have been already been added, i.e., media obtained from ongoing cell culture experiments, or in other embodiments, be media prior to the addition of cells.

As used herein, the term “reagent” refers to any solution used in a laboratory or clinical setting for biomedical and molecular biology applications. Reagents include, but are not limited to, saline solutions, PBS solutions, buffer solutions, such as phosphate buffers, EDTA, Tris solutions, and the like. Reagent solutions can be used to create other reagent solutions. For example, Tris solutions and EDTA solutions are combined in specific ratios to create “TE” reagents for use in molecular biology applications.

### Target Component

As used herein, the term “target component” refers to any molecule, cell or particulate that is to be filtered, separated, and/or identified from a source fluid. Representative examples of target cellular components include, but are not limited to, mammalian cells, viruses, bacteria, fungi, yeast, protozoan, microbes, parasites, and the like. Representative examples of target molecules include, but are not limited to, pathogens, hormones, cytokines, proteins, peptides, prions, lectins, oligonucleotides, contaminating molecules and particles, molecular and chemical toxins, and the like. The target components also include contaminants found in non-biological fluids, such as pathogens or lead in water or in petroleum products. Parasites include organisms within the phyla Protozoa, Platyhelminthes, Aschelminithes, Acanthocephala, and Arthropoda.

As used herein, the term “molecular toxin” refers to a compound produced by an organism which causes or initiates the development of a noxious, poisonous or deleterious effect in a host presented with the toxin. Such deleterious conditions may include fever, nausea, diarrhea, weight loss, neurologic disorders, renal disorders, hemorrhage, and the like. Toxins include, but are not limited to, bacterial toxins, such as cholera toxin, heat-liable and heat-stable toxins of E. coli, toxins A and B of Clostridium difficile, aerolysins, hemolysins, and the like; toxins produced by protozoa, such as Giardia; toxins produced by fungi; and the like. Included within this term are exotoxins, i.e., toxins secreted by an organism as an extracellular product, and enterotoxins, i.e., toxins present in the gut of an organism.

In some embodiments, the target component is a bioparticle/pathogen selected from the group consisting of living or dead cells (prokaryotic and eukaryotic, including mammalian), viruses, bacteria, fungi, yeast, protozoan, microbes, parasites, and the like. As used herein, a pathogen is any disease causing organism or microorganism.

Exemplary mammalian cells include, but are not limited to, stem cells, cancer cells, progenitor cells, immune cells, blood cells, fetal cells, and the like.

Exemplary fungi and yeast include, but are not limited to, Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii (or Pneumocystis carini\ddot{ι}), Stachybotrys chartarum, and any combination thereof.

Exemplary bacteria include, but are not limited to: anthrax, Campylobacter, cholera, diphtheria, enterotoxigenic E. coli, giardia, gonococcus, Helicobacter pylori, Hemophilus influenza B, Hemophilus influenza non-typable, meningococcus, pertussis, pneumococcus, salmonella, shigella, Streptococcus B, group A Streptococcus, tetanus, Vibrio cholerae, yersinia, Staphylococcus, Pseudomonas species, Clostridia species, Myocobacterium tuberculosis, Mycobacterium leprae, Listeria monocytogenes, Salmonella typhi, Shigella dysenteriae, Yersinia pestis, Brucella species, Legionella pneumophila, Rickettsiae, Chlamydia, Clostridium perfringens, Clostridium botulinum, Staphylococcus aureus, Treponema pallidum, Haemophilus influenzae, Treponema pallidum, Klebsiella pneumoniae, Pseudomonas aeruginosa, Cryptosporidium parvum, Streptococcus pneumoniae, Bordetella pertussis, Neisseria meningitides, and any combination thereof.

Exemplary parasites include, but are not limited to: Entamoeba histolytica; Plasmodium species, Leishmania species, Toxoplasmosis, Helminths, and any combination thereof.

Exemplary viruses include, but are not limited to, HIV-I, HIV-2, hepatitis viruses (including hepatitis B and C), Ebola virus, West Nile virus, and herpes virus such as HSV-2, adenovirus, dengue serotypes 1 to 4, ebola, enterovirus, herpes simplex virus 1 or 2, influenza, Japanese equine encephalitis, Norwalk, papilloma virus, parvovirus B 19, rubella, rubeola, vaccinia, varicella, Cytomegalovirus, Epstein-Barr virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, poliovirus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Human T-cell Leukemia virus type-1, Hantavirus, Rubella virus, Simian Immunodeficiency viruses, and any combination thereof.

Exemplary contaminants found in non-biological fluids can include, but are not limited to microorganisms (e.g., Cryptosporidium, Giardia lamblia, bacteria, Legionella, Coliforms, viruses, fungi), bromates, chlorites, haloactic acids, trihalomethanes, chloramines, chlorine, chlorine dioxide, antimony, arsenic, mercury (inorganic), nitrates, nitrites, selenium, thallium, Acrylamide, Alachlor, Atrazine, Benzene, Benzo(a)pyrene (PAHs), Carbofuran, Carbon, etrachloride, Chlordane, Chlorobenzene, 2,4-D, Dalapon, 1,2-Dibromo-3-chloropropane (DBCP), o-Dichlorobenzene, p-Dichlorobenzene, 1,2-Dichloroethane, 1,1-Dichloroethylene, cis-1,2-Dichloroethylene, trans-1,2-Dichloroethylene, Dichloromethane, 1,2-Dichloropropane, Di(2-ethylhexyl) adipate, Di(2-ethylhexyl) phthalate, Dinoseb, Dioxin (2,3,7,8-TCDD), Diquat, Endothall, Endrin, Epichlorohydrin, Ethylbenzene, Ethylene dibromide, Glyphosate, Heptachlor, Heptachlor epoxide, Hexachlorobenzene, Hexachlorocyclopentadiene, Lead, Lindane, Methoxychlor, Oxamyl (Vydate), Polychlorinated, biphenyls (PCBs), Pentachlorophenol, Picloram, Simazine, Styrene, Tetrachloroethylene, Toluene, Toxaphene, 2,4,5-TP (Silvex), 1,2,4-Trichlorobenzene, 1,1,1-Trichloroethane, 1,1,2-Trichloroethane, Trichloroethylene, Vinyl chloride, and Xylenes.

### Assay

The invention also provides a method of identifying at least one target component in a source fluid, the method comprising: mixing a plurality of magnetic microbeads with the source fluid to enable binding of the at least one target component to one or more magnetic beads, wherein surface of the magnetic beads is funcationalized to include at least one binding molecule that can bind with the target component in the fluid; flowing the source fluid through a microdevice described herein; exposing the source fluid containing at least one magnetic microbead bound target component to a magnetic field gradient positioned to cause the magnetic microbead bound target component to migrate into the capture chamber; and detecting and/or analyzing at least one of the magnetic microbead target components in the capture chamber.

The amount of source fluid used in an assay described herein assay will depend on factors such as the microdevice dimensions, flow rate, time constrains, and the concentration of the target component in the source fluid. Accordingly, amount of source fluid to be passed through the microdevice can range from 1 ml to 1 L. In some embodiments, from about 1 ml to about 500 ml, or about from 5 ml to about 250 ml, or from about 7.5 ml to about 100 ml of source fluid can be passed through the microdevice. In one embodiment, about 10 ml of source fluid can be passed through the microdevice.

A source fluid sample can be pre-treated before mixing of magnetic microbeads. For example, a biological sample can be pre-treated to inhibit activity of one or more enzyme present in the biological fluid, inhibit coagulation, make the sample more amenable to flowing through the device etc.

In some embodiments, the method further comprises the step of providing a plurality of microbeads, wherein surface of the magnetic beads is functionalized to include at least one binding molecule that can bind with the target component in the fluid.

The amount of magnetic beads added to the sample depend on a number of factors including number of binding molecules present on a single magnetic microbead, size of the magnetic beads, detection method being used, target component to be identified, and concentration of the target component in the source fluid. Accordingly, in some embodiments, from about 10 to 10⁶ of magnetic microbeads are mixed with 1 ml of source fluid sample.

Generally, the source fluid is passed once through the microdevice. However, the source fluid can be collected at the outlet and passed through the device again as needed. Thus, a single sample of source fluid can be passed through the device 1, 2, 3, 4, 5, 6, 7, 8, 9, 10 or more times.

In one embodiment, the method also includes the step of obtaining a source fluid sample. For example, obtaining a biological fluid sample from a subject. The skilled artisan is well aware of the methods for obtaining biological fluid samples from a subject including drawing blood and obtaining urine samples.

After the source fluid has passed through the device, the device can be washed by flowing an appropriate fluid, e.g., a washing fluid such as a buffer, through the microchannels. According, in some embodiments, the method further comprises the step of flowing a fluid, such as a buffer, through the microdevice. Amount of fluid to be flown through the microdevice can be any amount and can be based on the volume of the source fluid. In some embodiments, amount of the washing fluid is from about 0.5× to about 10× volume of the source fluid. In one embodiment, amount of the washing fluid is from about 1.5× to about 2.5× volume of the source fluid. In some embodiments, mount of the washing fluid is from about 0.5× to about 10× total volume of the microchannels in the device. In one embodiment, amount of the washing fluid is from about 1.5× to about 2.5× total volume of the microchannels in the device.

### Detection of Bound Target Component

A detection component, device or system can be used to identify the presence of the separated target component by optical, electrical, electrochemical, or other means. Detection, such as pathogen detection, can be carried out using light microscopy with phase contrast imaging based on the characteristic size (5 um diameter), shape (spherical to elliptical) and refractile characteristics of target components such as pathogens, for example, in the case of fungi that are distinct from all normal blood cells. Greater specificity can be obtained using optical imaging with fluorescent or cytochemical stains that are specific for all pathogens or specific subclasses (e.g. calcofluor (1 μM to 100 μM) for chitin in fungi, fluorescent antibodies directed against fungal surface molecules, gram stains, acid-fast stains, etc.).

Pathogen detection can also be carried out using an epifluorescent microscope to identify the characteristic size (5 um diameter), shape (spherical to elliptical) and staining characteristics of pathogens. For example, fungi stain differently from all normal blood cells, strongly binding calcofluor (1 μM to 100 μM) and having a rigid elliosoid shape not found in any other normal blood cells.

For optical detection, including fluorescent detection, more that one stain or dye can be used to enhance the detection and/or identification of the target component. For example, a first dye or stain can be used that can bind with a genus of target component, and a second dye or strain can be used that can bind with a specific target component. Colocalization of the two dyes then provides enhanced detection and indentification of the target component by reducing false positive detection of target components.

The stains and dyes can be stored in a separately micromolded reservoir within the diagnostic microdevice and pumped through the CVC or the capture and visualization region after the magnetic separation process is complete to stain the collected cellular components. Because the CVC can be connected to all flow channels, magnetically separated pathogens can be stained and imaged simultaneously. The clear PDMS ceiling of the CVC and the capture and visualization region allows visual examination of stained pathogens; however, other clear biocompatible materials can be used for this purpose, and non-clear materials can be utilized when other types of detection components (e.g., optical resonance detectors) in the system. In addition, the magnet can also be removed, and then the stained cells can be repositioned or collected together in the CVC or the capture and visualization region using, for example, a smaller permanent magnet positioned at one localized site to further concentrate the rare pathogens to improve imaging or detection sensitivity for rare components, and to collect and/or store them for subsequent culture and sensitivity testing. Alternatively, an electro-magnet can be controlled to concentrate the pathogens at one localized site.

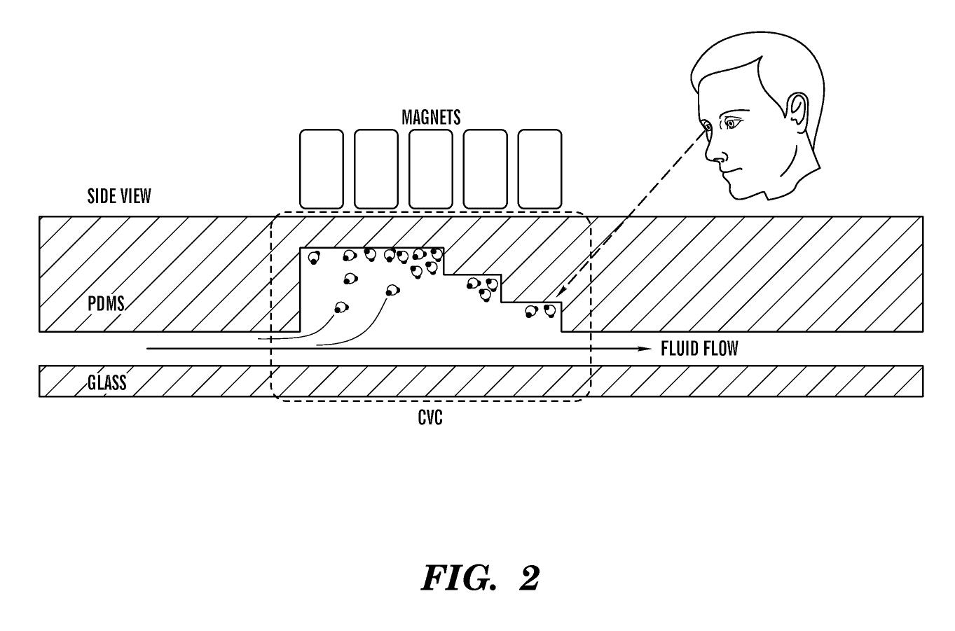

In some cases, magnetic collection of beads and bound pathogens within the microfluidic device can result in a dense grouping of the magnetic beads, which are not translucent and hence this can obscure low frequency pathogens from the view when visual or direct optical detection methods are utilized. This dense grouping of magnetic microbeads can be separated by applying magnetic field configurations that induce magnetic beads to generate inter-bead forces in liquids that cause them to separate into evenly distributed arrays with spaces between each microbead. As shown in FIG. 3, this can be accomplished by first collecting the magnetic beads and bound pathogens from the device in a small drop (˜0.5 mL) of liquid (e.g., isotonic saline, water), and then using a flat ring-shaped rare earth magnet with a similarly shaped magnetic field gradient concentrator composed of magnetic steel to magnetically induce a regularly arrayed pattern of beads in a geodesic (minimum path) distribution on the surface of the water droplet. The shape of this distribution can be varied by altering the magnetic configuration.

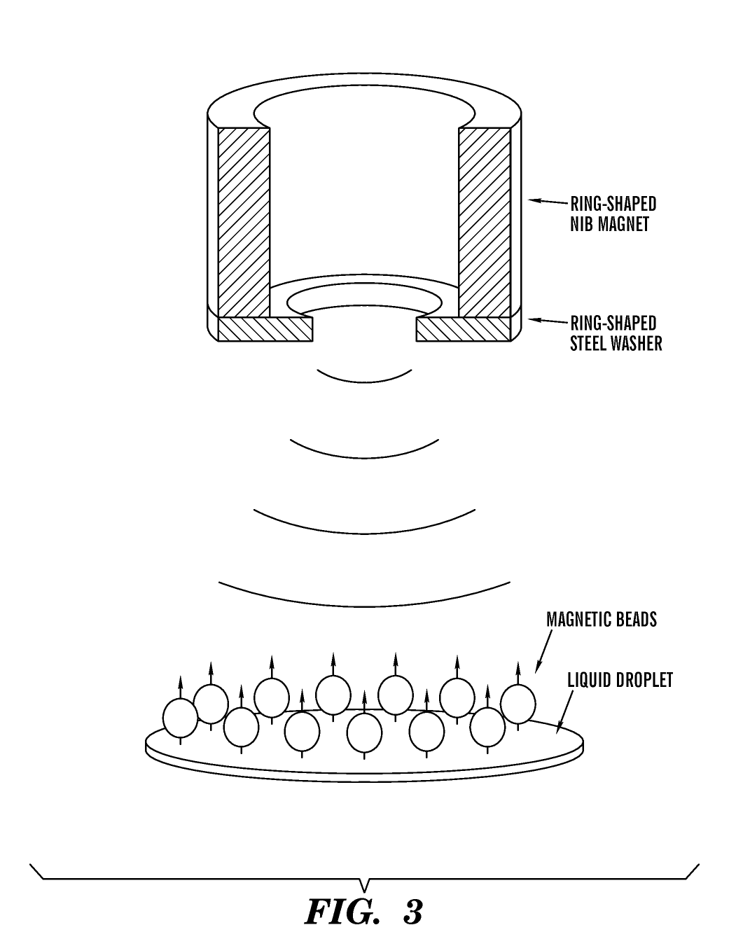

The assembly of the geodesic magnetic bead array is driven by a combination of surface tension forces and the paramagnetic nature of the magnetic beads employed for the diagnostic assay. In the presence of an external magnetic field, the super-paramagnetic core of each bead becomes magnetized and experiences an attractive force parallel to the field lines of the external magnetic field. As shown in FIG. 4A, the vector lines of the external magnetic field are oriented perpendicular to the surface of the liquid so that the bead suspension is attracted up to the air-liquid surface and is then held there by a combination of surface tension and magnetic attraction. FIG. 4B shows the magnitude of the magnetic field in the vertical direction within the plane of interest. For the size of the beads used in this embodiment of the invention (preferably, 1 to 10 um; potentially 0.1 to 50 um), the upward force produced by the magnetic field is balanced by the downward force generated by the surface tension to retain the hydrophilic beads at the air/liquid interface (in this embodiment, air/water, but it could be interfaces, such as isotonic saline as well). A similar separation might be accomplished in a closed microfluidic system by pulling the beads to a liquid/liquid interface between water and a biocompatible oil (e.g., pharmaceutical, cosmetic or food-grade mineral oils, etc.).

The formation of the geodesic array can occur when the external magnet field produces a small magnetic dipole in each of the beads along the same vector as the local external field, also perpendicular to liquid surface. The interactions between these induced dipoles are repulsive, serving to spread the beads apart on the surface in much the same way as when two parallel bar magnets are brought into close proximity. The geodesic array minimizes the energy of the system by maximizing the distance between the beads. Holding the beads at the liquid/air interface places them in a low friction environment where these weak repulsive forces can affect the system because the liquid will continuously shear under any applied force. This same repulsive phenomenon also exists when beads are held at solid/air interface by an external field, but usually they are much too weak to overcome the friction in solids, such as glass or PDMS found in the cell separation microfluidic device. The spacing between the beads depends on the strength of the induced magnetic dipole in each bead core. The strength of these dipoles is directly proportional to the external magnetic field, which can be altered to tune the parameters of the system.

In accordance with one embodiment of the invention, a neodymium ring magnet combined with a ring-shaped steel washer as shown in FIG. 3 can be used to produce a suitable field to make geodesic magnetic bead arrays in order to enhance pathogen visualization. The magnet can be used to produce a strong magnetic dipole perpendicular to the plane of the magnet that passes through the center of the central aperture as shown in FIG. 4A. The body of magnet can be positioned parallel to the surface of the liquid so that its net dipole is also perpendicular to the liquid surface plane of interest. As shown in FIGS. 4A and 4B, numerical simulations using COMSOL show that the high magnetic permeability of the steel washer redirects the magnetic field to maximize the vertical component of the field used to create the array while minimizing the radial component of the field, which tends to pull the beads outwards along the liquid surface to edges of the liquid.

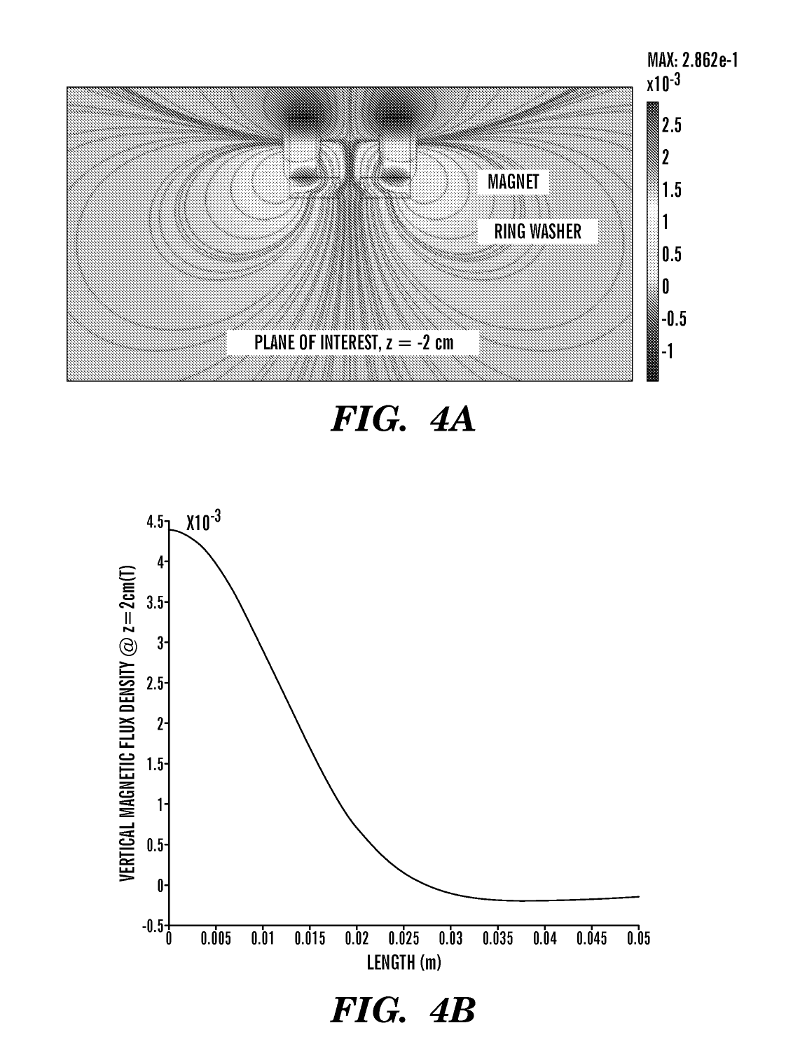

In accordance with one embodiment of the invention, a neodymium ring magnet and washer can be use to form geodesic magnetic bead arrays on water drops positioned at a distance (˜2 cm away from the magnet) as shown in FIG. 5. The array was made with 1 um beads, the induced geodesic pattern covered approximately a 0.5 cm diameter area. Various sizes of ring washers can be used to help realign the magnetic field to maximize vertical field vectors directly below the magnet and minimize the radial field component. However, the results shown in FIG. 5 demonstrate that separated beads can be uniformly disperse on the surface of water droplets to maximize visualization for even photometric detection, as previously demonstrated by our laboratory with larger (4.5 um) magnetic beads. The present invention can also be used with beads bound to fungal pathogens isolated from blood using the microfluidic cell separation device.

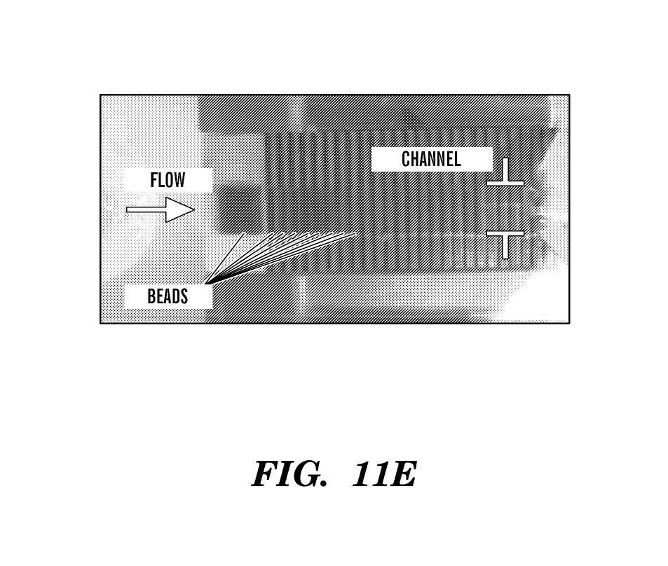

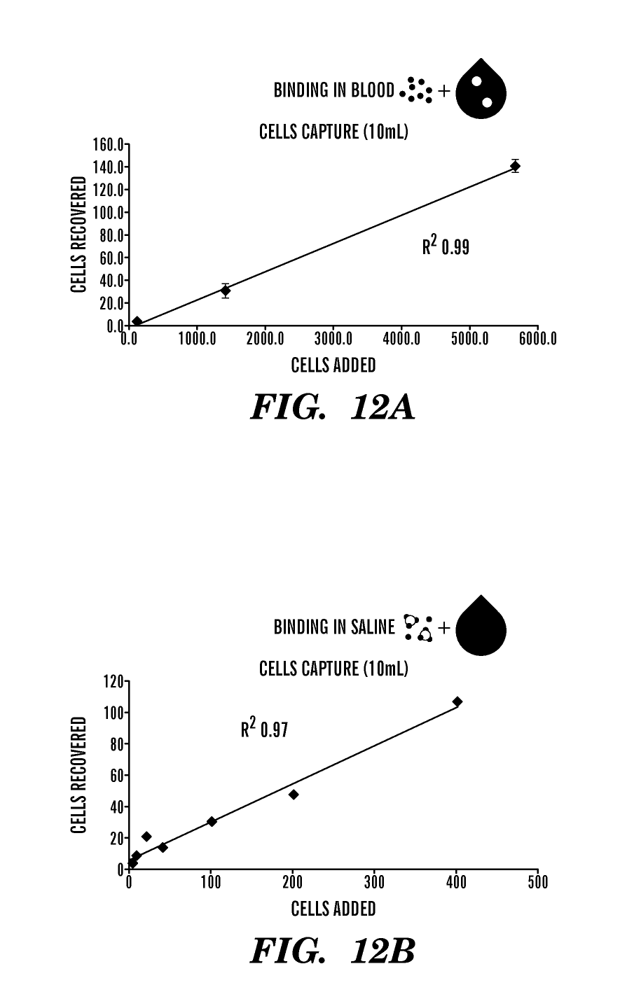

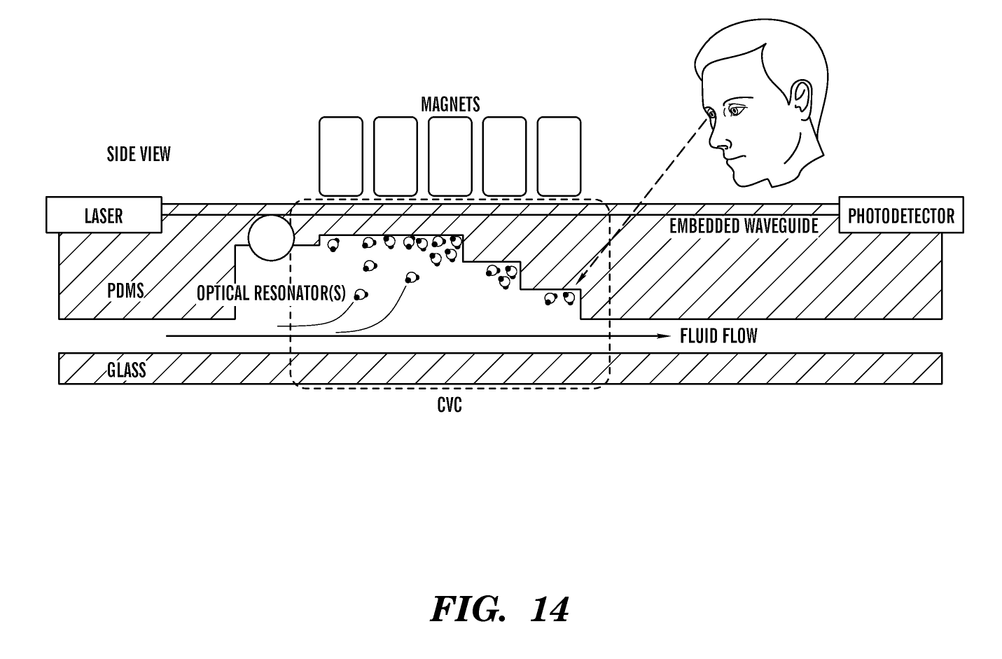

In one embodiment, the detection is by a high-Q optical resonator as described in U.S. Prov. App. No. 61/296,357, filed Jan. 19, 2010. Detection by a high-Q optical resonator can be illustrated as follow. The magnetic micro- or nanoparticles are used to remove the pathogens or biomarkers from the remainder of the flowing blood using a magnet (or an electro-magnet) contained within the device housing as shown, for example, in FIG. 14 f. Saline that can be pre-packed in another microchamber in the device will be flowed through the channel to wash the collected micro-nanoparticles that now carry the pathogen or biomarker free of blood components, and then the magnet will be moved away from the channel to release the magnetic micro- or nanoparticles and bound pathogen or biomarkers so that they flow into another channel where they pass over a series of solid-state microfabricated silicon optical resonators (driven by inexpensive chip-scale laser diodes), each containing a genetically engineered ligand for surface molecules expressed in a specific manner by each class of pathogen (e.g., virus, fungus, protozoan, gram-negative bacteria, gram-positive bacteria) that warrants a different class of antibiotic therapy. Binding of the micro- or nanoparticles that carries a pathogen or biomarker to the surface of the appropriate resonator as shown, for example, in FIG. 15. will alter the optical resonance at that site, leading to a change in signal output and can be indicated in the form of an LED readout that delineates the pathogen class, and hence, informs the caregiver to triage the patient appropriately (e.g., transfer them from a doctor's office to a hospital) and to initiate a particular type of antimicrobial therapy. In another scheme, the magnetic micro- or nanoparticles are collected and immobilized using a static magnetic field. The pathogens, or biomarkers or parts thereof are then released from the micro- and nanoparticles and detected in a label-free manner using an optical resonator located near-by as shown, for example, in FIG. 17. Without wishing to be bound by a theory, individual size and shape of bound micro- or nanoparticles can be determined from the magnitude of the frequency shifts in optical resonators.

In one embodiment, the optical resonator itself can be made specifically for detection of magnetic micro- or nanoparticles that carry target analyte by immobilizing one or more recognition elements such as antibodies directly to the resonator surface. In this scheme, the presence of target analyte is detected in real-time from the frequency shift of the optical resonator as magnetic micro or nanoparticles, only those that carry target analyte, bind to the recognition elements on the resonator surface as shown, for example, in FIG. 15.

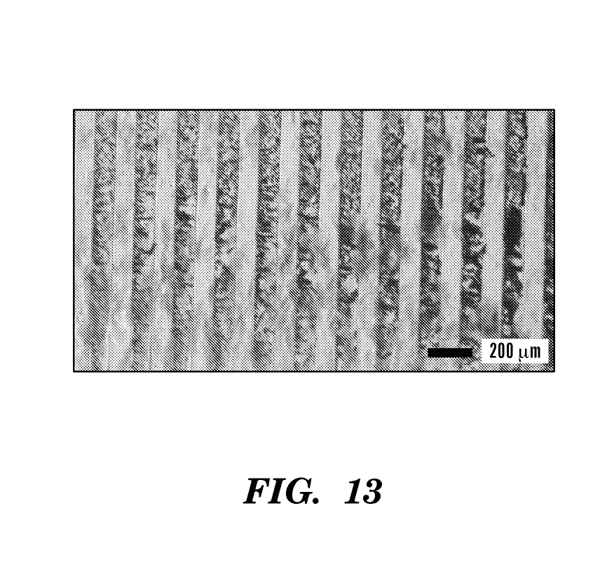

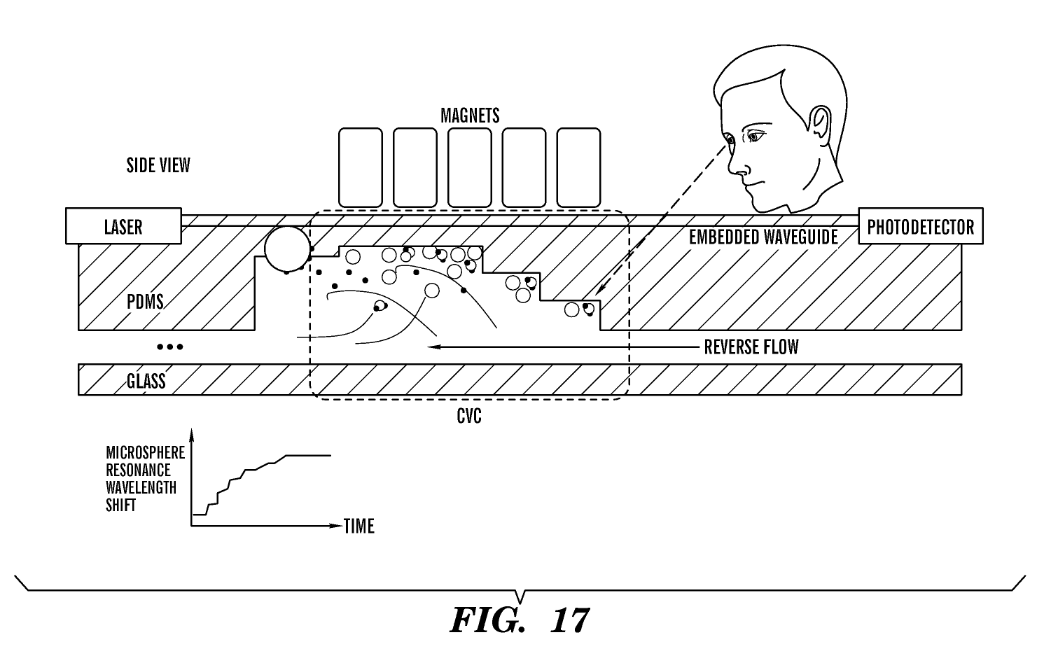

Detailed description of the sensing scheme is shown in FIG. 19. The optical resonator comprises a ˜100 um-diameter silica microsphere or a plurality thereof where each microsphere is coupled to the same or a separate optical waveguide. An optical signal is generated by coupling the output of a tunable laser to one end of the waveguide, for example by using a free space fiber coupler. Alternatively, the laser can be directly coupled to the optical waveguide by using a ‘fiber pigtail’. Examples for tunable lasers are inexpensive distributed feedback (DFB) laser, chip-scale devices that operate in the telecom band at ˜1550 nm or ˜1310 nm nominal wavelength. The optical waveguide may comprise a standard smf-28 single mode optical fiber which has been tapered in its midsection where the fiber makes contact with the microsphere sensor. The tapered fiber region allows the light to couple from the fiber to the microsphere, where the light then stays confined due to total-internal reflection, on an orbital trajectory close to the microsphere surface as shown, for example, in FIG. 16A. Since the trapped lightwave inside the microsphere has to return in phase for each roundtrip in order to avoid destructive interference, the rerouting of light from the fiber to the microsphere occurs only for that wavelength which fulfills this resonance condition as shown, for example, in FIG. 16A. This specific resonance wavelength of the microsphere is identified by tuning the wavelength of the DFB laser. If the wavelength of the laser is identical to the resonance wavelength of the microsphere, the light no longer reaches the photodetector located at the other end of the fiber and instead couples to the microsphere as shown, for example, in FIG. 16B, top. A drop is recorded in the transmission spectrum, the minimum of which corresponds to the precise resonance wavelength. Measurement of this resonance wavelength provides a label-free means for detection of particles. The binding of a micro- or nanoparticle to the microsphere surface causes a change in the resonance wavelength and binding events are detected in real-time by tracking the precise change of the resonance wavelength as shown, for example, in FIG. 16B, bottom. Specific detection of analyte is possible if recognition elements are pre-immobilized on the microsphere surface as shown, for example, in FIGS. 15 and 17, examples of recognition elements are antibodies, lectins, bacterial membranes, etc. To immobilize the recognition elements, the surface of the optical resonator, in this example silica, can be modified by chemical compounds that covalently bind to silica's silanol groups. A prominent chemical for this purpose is aminosilane, a bifunctional molecule which provides reactive amino groups after silanol-linkage to the glass surface. The amino-groups are used to conjugate to activated carboxyl groups of peptide or carbohydrate moieties of the recognition elements. Previous to sensing, magnetic micro-or nanobeads are used to collect and concentration the analyte by optimal exposure of magnetic beads to the sample, where the magnetic beads are chemically modified and carry biorecognition elements that bind to pathogens or biomarkers as shown, for example, in FIGS. 15 and 17. After exposure, the magnetic micro- and nanobeads are collected with a magnet. In one embodiment as shown, for example, in FIG. 15, the beads are then released by removing the stationary magnetic field, and beads that carry pathogen or biomarker are identified from specific binding to optical resonators which have been previously modified with the same or different recognition element. In another embodiment ,as shown in FIG. 17, the magnetic beads remained trapped, and instead the analyte is released from the surface of the micro- or nanobeads for example by introducing a chemical releasing agent with a (here reversed) microfluidic flow as shown in FIG. 17. Binding of the released analyte is then detected directly (label-free) from resonance wavelength shift that occur as the analyte binds to recognition elements on the optical resonator surface (here a glass microsphere). The analyte could be example cancer marker CA 19-9, CEA, virions, HIV, Influenza A, fungi, components of fungi cell wall, etc. Other chemical agents may be introduced not only to release the analyte but also to modify the analyte. For example, chemical agents may be introduced that lyse fungi/cells/bacteria, that digest DNA/cell wall/proteins, or that disintegrate a lipid bilayer (SDS etc.).

Sensitive detection down to single micro- or nanoparticles is feasible by using a high Q optical resonator such as a silica microsphere. Silica microspheres can be simply fabricated by melting the tip of a standard single mode optical fiber using butane/nitrous oxide flame or a carbon-dioxide laser. Other examples for chip-based high-Q optical resonators are silicon microrings and silicon photonic crystal cavities, structures that are amenable to fabrication by photolithography using CMOS technology.

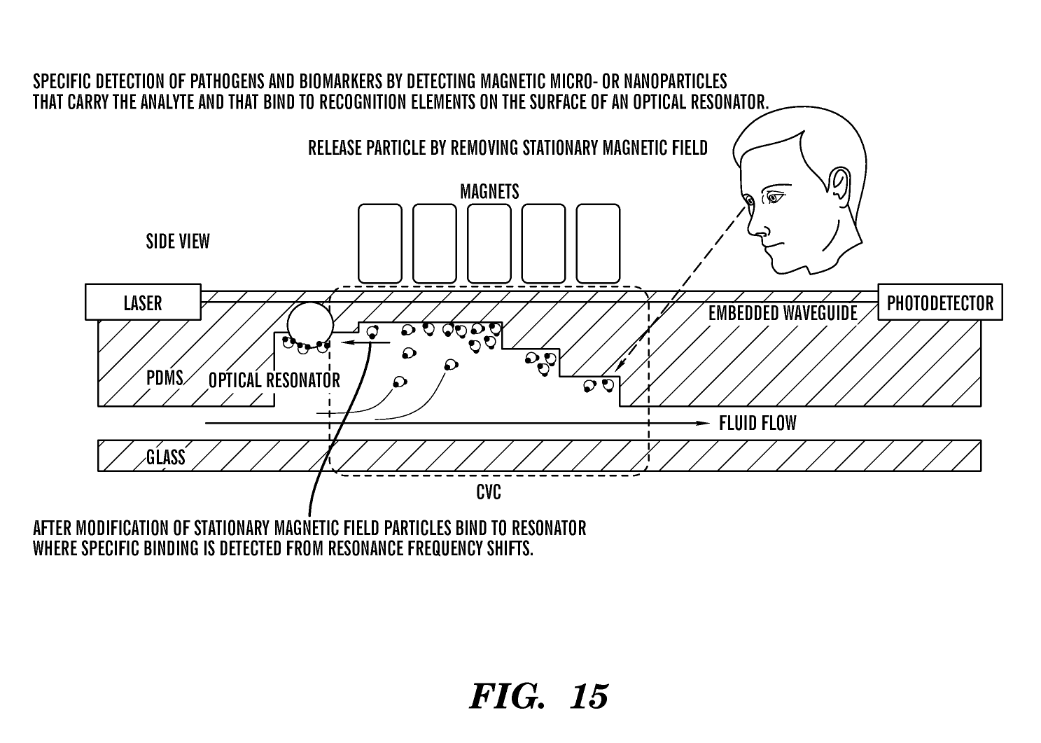

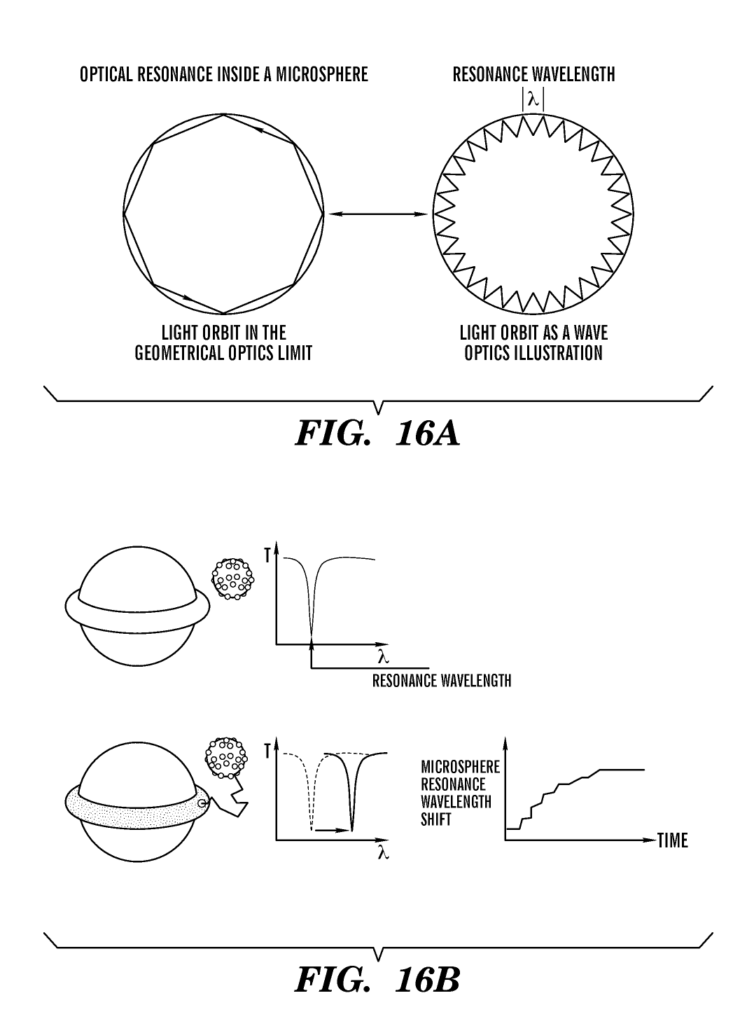

In another embodiment of the invention, the optical resonators themselves can be used to trap and concentrate the micro- and nanoparticles as shown, for example, in FIG. 18. Nanoparticles suspended in aqueous solution are normally in Brownian motion. However, within the reach of the optical resonator's evanescent field (˜200 nm) nanoparticles are drawn toward the surface by optical gradient forces, similar to those present in optical tweezers. The gradient forces draw the nanoparticles towards the high-intensity region of the evanescent field from where they tend to adsorb and accumulate on the surface of the resonator as shown, for example, in FIG. 18 (an example of a toroidal resonator). In the case of a low binding-affinity or a low density of binding sites, the nanoparticles even propel around the orbit by radiation pressure. Within this orbital trap, radial stochastic motion is induced by thermal energy within the exponential-potential-well setup by the evanescent field, forcing a nanoparticle to visit the surface many times per micron during its circumnavigation. As a result binding is essentially assured once the nanoparticle is pulled into this stochastic orbit. This considerably increases the binding rate even in the presence of very few binding sites and at extremely low nanoparticle concentrations (fM). In addition the nanoparticle is drawn to the highest intensity of the WGM where its presence produces the largest sensing signal (i.e. wavelength shift). We find that the optical binding energy W_{b} is proportion to the product of the resonant quality factor Q and the laser power P. Surprisingly, the threshold power for virus-sized particles is in the one hundred microwatt range due to the build up in intensity caused by the high Q of our WGMs (˜10⁷). Thermal energy plays the major combative role in trapping. The trap is secured by raising the binding energy W_{b} associated with the radial gradient force by a few times the Boltzman energy, k_{B}T. In the presence of appropriate antibodies at low densities on the surface, the bio-particle binding probability is extremely high. The beauty of this mechanism is that it attracts the particles to the largest intensity within the optical resonator orbit, which increases their concentration at the place of maximum sensitivity

In another embodiment of the invention, the optical detector component derives its high sensitivity for label-free detection from the use of optical resonance in glass microspheres, which is created when coherent light confined within the microsphere interferes constructively as shown, for example, in FIG. 19. Because these optical resonators are immune to damping in a liquid, they can be used as ultra-sensitive biosensors: for example, the sensor can detect binding of a single Influenza A (InfA) virion (100 nm) in an aqueous sample based on discrete resonance frequency-shifts without requiring any chemical or fluorescent labeling of the particles. Importantly, optical resonator components are not only highly sensitive, they also provide a versatile detection platform technology. The optical resonator sensors can be fabricated in various geometries (e.g., spheres, rings, capillaries, toroids, photonic crystals) and out of different optical materials, (e.g., glass, polymer, silicon wafers; using photolithographic techniques that facilitate mass-production of component parts at low cost. Rapid single particle detection is particularly relevant for diagnosis of viral infections that are capable of rapidly spreading through populations across the globe (InfA, SARS), or that suppress the immune system (e.g., HIV), because conventional detection assays are slow, expensive and require complicated equipment only available in hospital or commercial microbiological laboratories. Higher sensitivity of our optical resonator component will be required for detection of small HIV and HPV (˜50 nm) virions, and one can improve the sensor transduction mechanism by reducing cavity size. In accordance with the invention, optical resonators can be rendered virion-specific by conjugating biorecognition elements, such as specific antibodies, directly to the sensor surface, and the specificity can be increased via entirely optical means by quantizing resonance frequency shifts to determine virus particle size (InfA) and shape. Multiplexed analysis can be used to improve detection capabilities with enhanced specificity even in complex fluids such as blood, saliva and urine.

To optimize the technique, a system according to the invention can utilize 250 nm-radius polystyrene particles (PS) dissolved at femto-molar concentration in a drop of phosphate buffered solution (PBS) that surrounds a microsphere cavity. Whispering gallery mode (WGM) resonances are excited in the microsphere by evanescent coupling from a tapered single mode optical fiber. A transmission spectrum is recorded while the wavelength of a distributed feedback laser is tuned across one or more WGMs. The resonance wavelength is determined from the transmission spectrum by locating the minimum of a Lorentzian-shaped resonant line and then plotted versus time with ˜10 ms resolution.

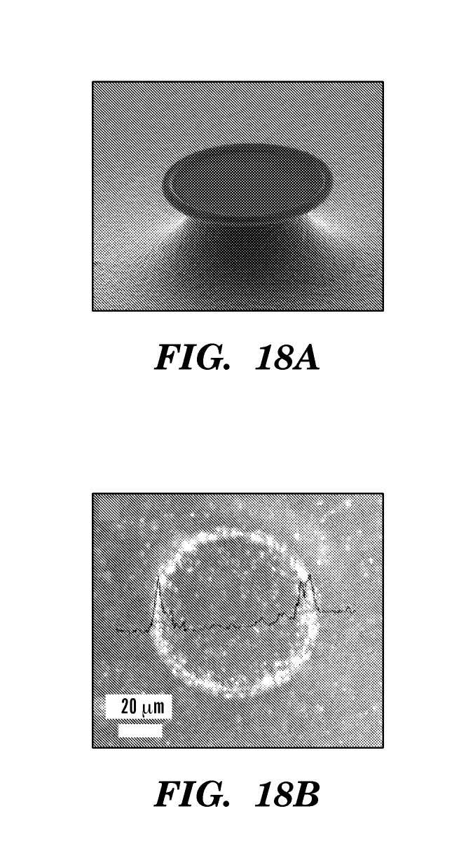

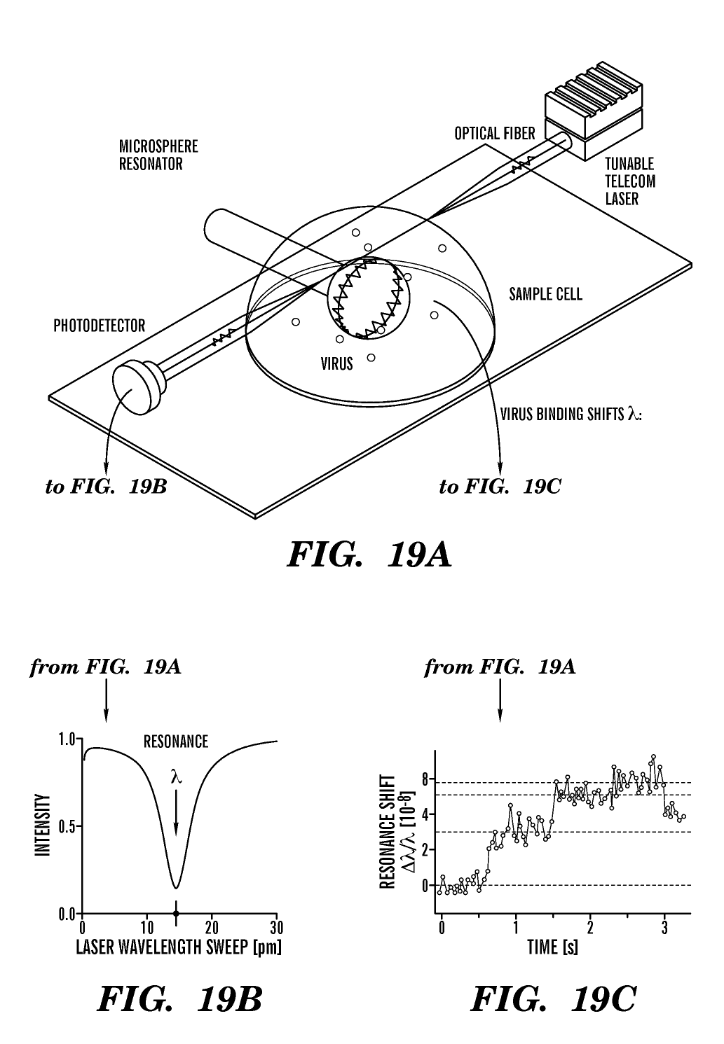

Microspheres can be fabricated from thinned optical fiber ends that are melted in a focused 10 W CO₂ laser. Immediately after its fabrication, the microsphere-on-a-stem structure is mounted on the sample cell. The sample cell is enclosed to limit air flow and stabilize the ambient humidity level as well as temperature. FIG. 20A shows a trace of the recorded fractional resonance wavelength change Δλ/λ for radius a=250 nm PS particles interacting with a microsphere cavity with radius R˜27 μm. Spikes of various heights are clearly visible against the background cavity noise indicating cavity perturbations by individual PS particles. A single binding and unbinding event can be discerned from the step in the wavelength shift signal close to the 300 second time point (binding) and close to the 400 second time point (unbinding). A maximum spike amplitude/step height can be distinguished by plotting a histogram of all events (not shown). Each wavelength shift induced by a PS nanoparticle is associated with a change in the resonance line-width (quality Q-factor) due to the scattering induced by the particle. The simultaneously recorded change in line-width is shown in FIG. 20B, for example.

The signal shift can be optimized by reducing microsphere size. In accordance with the invention, different-sized microspheres (R=44 μm−105 μm) can be used and a strong dependence of the fractional wavelength shift on the cavity radius, scaling as ˜R^{-2.5}. This is in good agreement with electromagnetic theory associated with single particle reactive WGM sensing, where the largest step heights are predicted for equatorial binding events. In contrast, a ˜1/R dependence is expected for a shift due to a random surface density. Similarly, the sensitivity of silicon ring resonators and silicon photonic crystal resonators will depend on cavity size (mode volume).The analysis of the wavelength shift signal is carried out for the case of a microspherical cavity. Single particle detection with microcavities relies on the fact that work is done by the evanescent field of a microcavity as the nanoparticle moves from a distant position to the microcavity surface. As a result the energy of light in the resonator is reduced. With the number of microcavity photons conserved, the frequency of each photon is shifted by Δωᵣ in accordance with

$$ℏΔ {ω}_{r} ≅ - \frac{{α}_{ex}}{2} 〈 {E ( {r}_{v} , t )}^{2} 〉 , \qquad ( 1 )$$

where <E(rᵥ,t)²> is the time average of the square of the field amplitude at the nanoparticle's position rᵥ due to a single photon resonant state. We assume that the nanoparticle is small compared to the wavelength, and has an excess polarizability αₑₓ. By dividing the shift in frequency by the single photon energy ωᵣ on the left and by the volume integral of the associated electromagnetic energy density on the right, we derive a simple expressions for the fractional frequency shift,

$$( \frac{Δ {ω}_{r}}{{ω}_{r}} ) ≅ \frac{- ( {α}_{ex} / {ɛ}_{0} ) {\vert {E}_{0} ( {r}_{v} ) \vert}^{2}}{2 \int {ɛ}_{r} ( r ) {\vert {E}_{0} ( r ) \vert}^{2} d V} , \qquad ( 2 )$$

where E₀ is the electric field amplitude, and εᵣ(r) is the dielectric constant throughout the cavity. Some insights are arrived at by rearranging Eq. 2;

$$( \frac{Δ {ω}_{r}}{{ω}_{r}} ) ≅ \frac{- ( {α}_{ex} / 2 {ɛ}_{0} )}{2 \int {ɛ}_{r} ( r ) {\vert \frac{{E}_{0} ( r )}{{E}_{0} ( {r}_{v} )} \vert}^{2} d V} . \qquad ( 3 )$$

Although Eqn. 3 was constructed by thinking about a single photon state, it applies equally well to multiple photons in the same state, since the square modulus of the field ratio in the denominator is independent of the number of photons. On the right in Eqn. 3 there is a ratio of volumes. The numerator is proportional bio-particle-volume V_{bp}, while the denominator will be defined as the sensing-mode-volume Vₛₘ. As Vₛₘ is reduced in relation to V_{bp}, the shift grows. For a 3D structure such as a microspherical resonator with a particle binding at the equator, one may expect Vₛₘ to be proportional to R³, and therefore provide a large advantage for single nanoparticle detection as the radius is reduced. This insight, although approximate, is none-the-less almost correct.

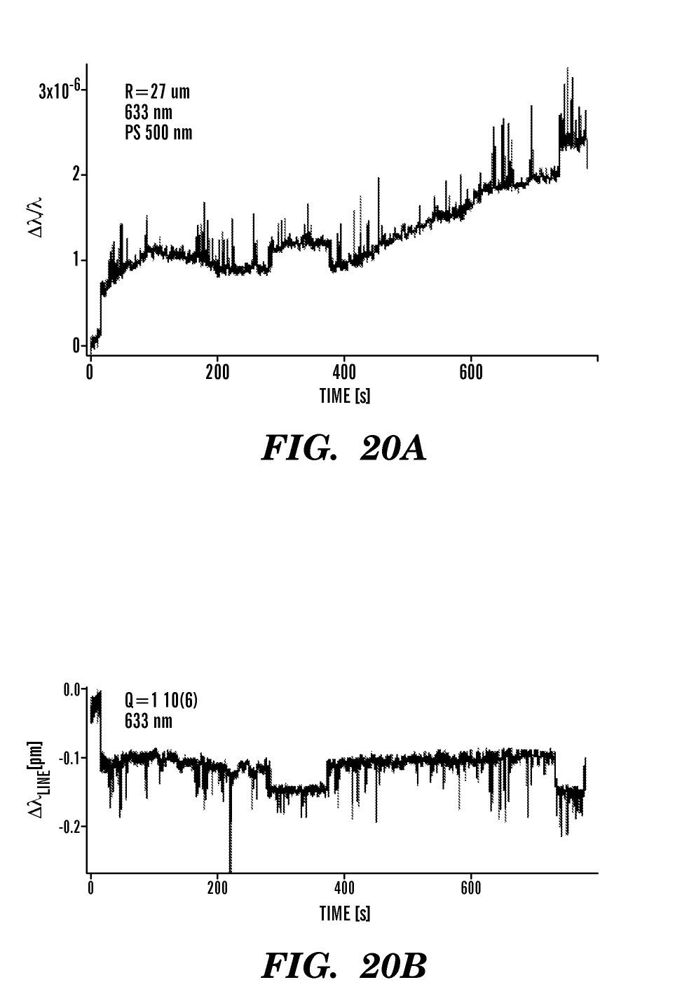

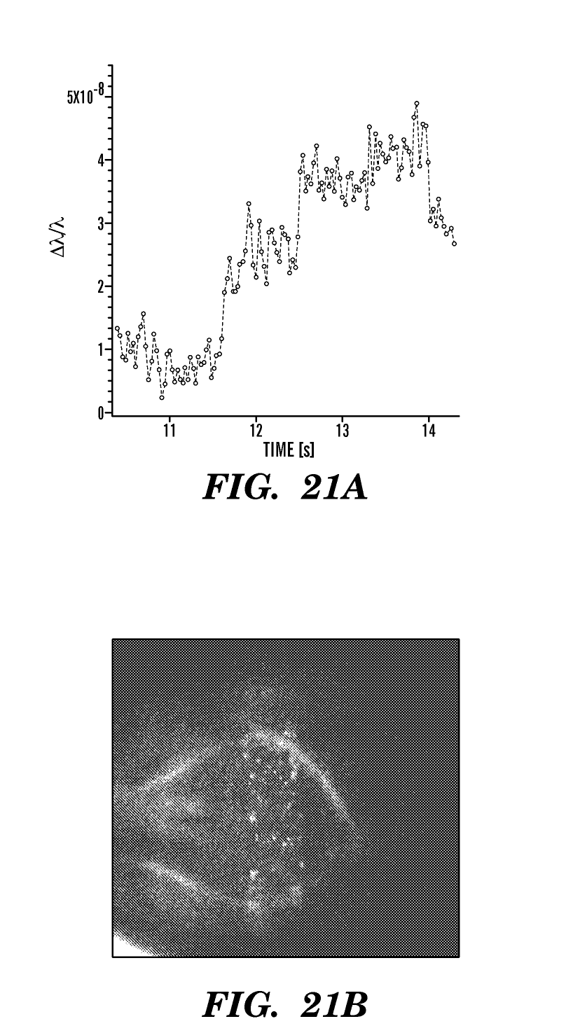

In accordance with the invention, the optical sensor utilizes the reactive sensing mechanism to increase the wavelength shift magnitude due to single nanoparticles by reducing microcavity size. Furthermore, the microsphere system can be optimized for the detection of a single Influenza A (InfA) virions. Following this approach, we use tunable laser with at λ˜763 nm wavelength and excite a WGM with Q˜6.4×10⁵ in R=39 μm microspheres. We inject InfA virions at concentration of ˜10 fM directly into a PBS filled sample cell, since the virions are known to adsorb to silica. The dip-trace of the resonance wavelength Δλ_{InfA}/λ in FIG. 21A reveals clear steps associated with binding of single viral particles. The signal-to-noise ratio (Δλ_{InfA}/Δλₙₒᵢₛₑ˜3) can be further improved upon by signal processing schemes such as Median filtering. In a second experiment, we label the InfA virus particles with DiIC membrane dye (invitrogen). Fluorescent images show the binding of InfA particles to the microsphere cavity (FIG. 21B). Surprisingly, we find most of the binding events to localize at the equatorial region where the principal photon energy resides. This observation indicates a novel optical mechanism for trapping and accumulation of nanoparticles by the optical field gradient of a microcavity—and may explain discrepancies for binding rates reported in the literature. Nanoparticles suspended in aqueous solution are normally in Brownian motion. However, within the reach of the WGM's evanescent field (˜200 nm) nanoparticles are drawn toward the surface by gradient forces, similar to those present in optical tweezers. The gradient forces draw the nanoparticles towards the high-intensity region of the evanescent field from where they tend to adsorb and accumulate on the surface of the resonator (FIG. 21, example for a toroidal resonator). In the case of a low binding-affinity or a low density of binding sites, the nanoparticles are propelled around the orbit by radiation pressure. Within this orbital trap, radial stochastic motion is induced by thermal energy within the exponential-potential-well setup by the evanescent field, forcing a nanoparticle to visit the surface many times per micron during its circumnavigation. As a result binding is essentially assured once the nanoparticle is pulled into this stochastic orbit. This considerably increases the binding rate even in the presence of very few binding sites and at extremely low nanoparticle concentrations (fM). In addition the nanoparticle is drawn to the highest intensity of the WGM where its presence produces the largest sensing signal (i.e. wavelength shift).

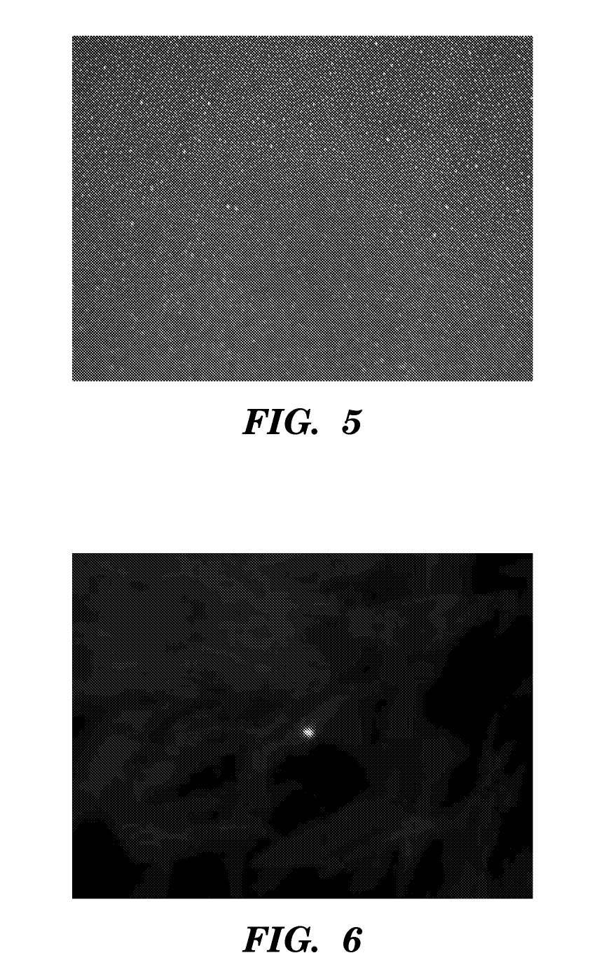

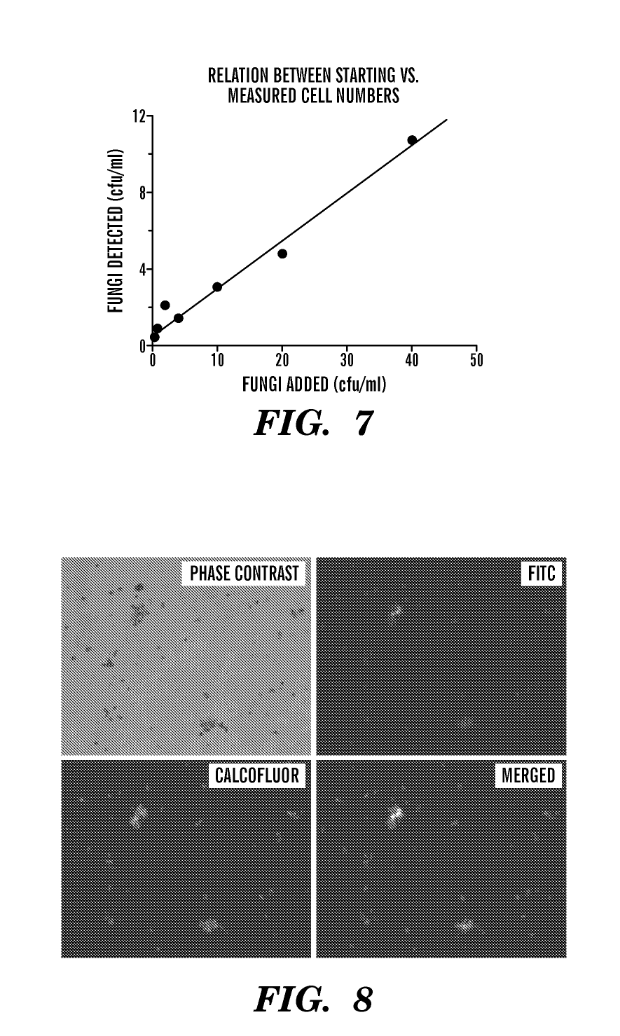

The device according to one embodiment of the invention can detect as few as one to two C. albicans fungal pathogens in 10 mL samples of human whole blood spiked with 0.4 cell colony forming units (cfu)/mL within ˜45 min after sample collection as shown in FIG. 6. Live C. albicans cells were bound by magnetic beads (1 um diameter) that were pre-coated with antibodies that bind to sugar groups (e.g. mannan) found on the surface of these cells, and then they were magnetically captured in a microfluidic device according to one embodiment of the invention. The captured cells can be fluorescently labeled by flowing the cellulose-binding Calcofluor (1 μm to 100 μM) dye through the microfluidic channel. As shown in FIG. 6, the captured cells can be easily distinguished among the many non-fluorescent beads using a conventional inverted fluorescent microscope (DAPI filter cube; 200× magnification). Using this approach as shown in FIG. 7, we could demonstrate a direct correlation between the number of fungal cells identified with the microfluidic device according to one embodiment of the invention and the concentration of pathogenic cells in the blood samples analyzed (because, in this case, known amounts of fungal cells were added to the blood). These data demonstrate the potential usefulness of this method for rapid (<45 min) diagnosis of blood-borne fungal infection, as well as quantitation of the fungal pathogen load in human blood samples.

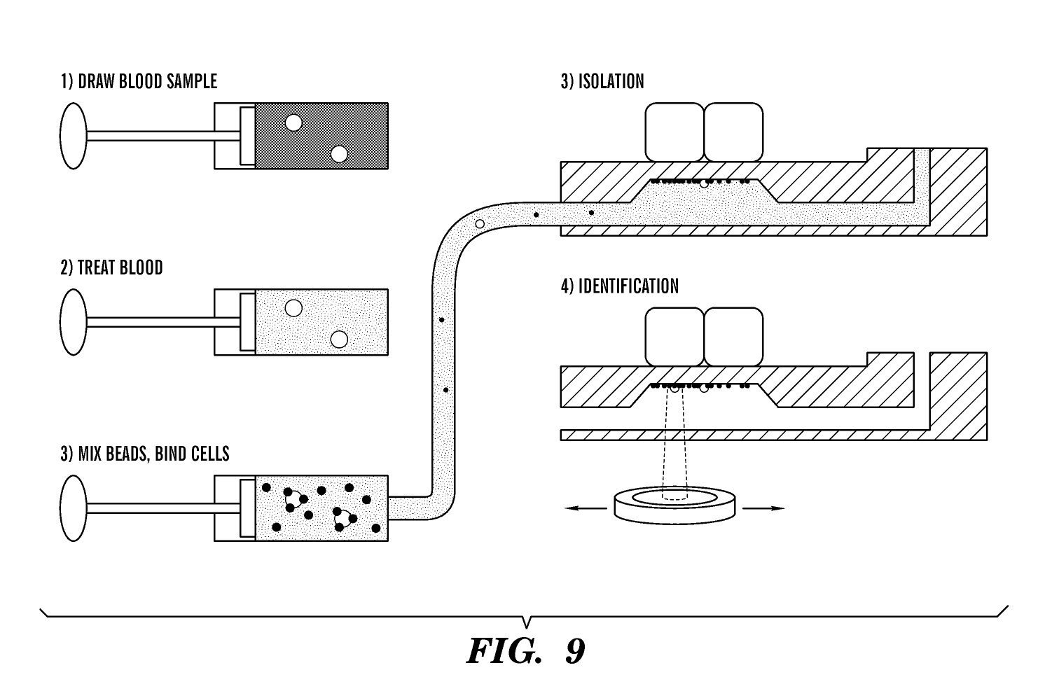

In addition to staining fungal cells with calcofluor (1 μM to 100 μM) dye, antibodies similar to those used to bind cells to magnetic beads may be conjugated with fluorophores (e.g. FITC) and used as a double stain. FIG. 8 shows C. albicans bound and separated with immunomagnetic beads that have been double stained with calcofluor (1 μM to 100 μM) and FITC conjugated antibodies. Simultaneous staining of C. albicans with calcofluor (1 μM to 100 μM) (blue—bottom left panel) and secondary FITC conjugated antibodies (green—top right panel). Fungi cells are first tagged and magnetically separated by immunomagnetic beads and then double stained by fluorescent calcofluor (1 μM to 100 μM) and antibody stains to confirm its identity. The lower-right panel of FIG. 8 shows the merged color image of the double stained cells.

The present invention includes a diagnostic device and associated technology that can be used for the general purpose of selectively detecting very low concentrations of any pathogens (bacteria, viruses, protozoans, as well as fungi), mammalian cells (e.g., cancer cells, fetal cells in maternal circulation, immune cells), infected cells (e.g., macrophages with injected microbes) or molecules (e.g., antibodies, cytokines, growth factors, hormones) present within various fluids that are otherwise undetectable or require time-consuming culture, analysis or bioassays to detect. The present invention includes diagnostic technology that provide platform that enables the rapid detection and diagnosis of wide variety of diseases, where each diagnosis can be customized based on the use of opsonins or ligands that are tailored to that disease. Opsonins used to bind specific particles of interest can include antibodies, as well as protein- or nucleotide-based aptamers, and antigen binding proteins, lectins (e.g., mannose binding lectin) or any other ligand for a surface component on the cell or molecule of interest. Techniques such as directed evolution and phage display can be used to further optimize specificity and strength of particle binding, in accordance with the invention.

Other embodiments of the present invention can, for example, include a simple, rapid and highly sensitive microfluidic device for pathogen detection that can be used as a point of care (POC) diagnostic, as well as a rapid detection and pathogen collection device in the hospital setting. One embodiment of the invention can be used to detect living C. Albicans pathogens that are a major cause of sepsis in humans, in whole human blood without requiring any pre-processing of blood. The high sensitivity provided by embodiments of the invention using simple fluorescent stains amenable to conventional fluorescent microscopes or LED detectors can be integrated on chip within these devices and enable the detection of less than one pathogen/mL of blood. As patients with systemic blood borne infections always have greater than 10 pathogen cells/mL of blood, and the great majority of patients have much higher levels (50 to hundreds of pathogen cells/mL), the present invention can be used to detect these pathogen with samples of less than 10 mL of human blood, which is easily accommodated for POC applications. Although the illustrated embodiment of the invention used fluorescent labels and microscopic detection to perform pathogen detection, alternative and even more sensitive detection components and devices, such as optical resonators, integrated into these devices (so that microscopes are not required), can be used to detect single, unlabelled viral particles 2 or electrochemical detectors. These devices in accordance with one or more embodiments of the invention can be easily sterilized and disposed of after use to minimize potential infection. These devices in accordance with one or more embodiments of the invention can be fabricated at low cost, can be simple to use, can provide high sensitivity, and can be used to preparatively isolate living pathogens that can be inserted into existing pathogen culture and sensitivity assays. These microdevices can have wide spread value as first stage pathogen diagnostics in both the community and hospital settings.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

Embodiments of the invention can be described by any of the following paragraphs:
1. A microfluidic device comprising:
   an inlet port adapted to be connected to a fluid source;
   an outlet port adapted to be connected to a fluid receiver;
   at least one microchannel connected to and extending between the inlet port and the outlet port;
   a capture chamber connected to the microfluidic channel, the capture chamber including at least one feature adapted to capture target components flowing in a source fluid provided by the fluid source; and
   a magnetic source disposed adjacent to the microchannel and configured to apply a magnetic field gradient to the source fluid flowing through the microchannel and to cause magnetic microbead bound target components in the source fluid to migrate into the capture chamber.
2. The microfluidic device of paragraph 1, wherein the microfluidic device further comprising a magnetic concentrator between the magnetic source and the microchannel.
3. The microfluidic device of paragraph 2, wherein the magnetic concentrator provides a uniform magnetic field gradient that extends into the microchannel, along the length of the capture chamber.
4. The microfluidic device of any of paragraphs 2-3, wherein the magnetic concentrator comprises a plurality of grooves on the surface adjacent to the microchannel.
5. The microfluidic device of any of paragraphs 2-4, wherein width of at least one groove is from about 0.1 μm to about 1000 μm.
6. The microfluidic device of any of paragraphs 2-5, wherein depth of at least one groove is from about 0.1 μm to about 2000 μm.
7. The microfluidic device of any of paragraphs 2-6, wherein space between the grooves is from about 0.1 μm to about 1000 μm.
8. The method of any of paragraphs 2-7, wherein the magnetic concentrator is fabricated from a material having high magnetic permeability.
9. The microfluidic device of any of paragraphs 1-8, wherein the microfluidic device comprises 1, 2, 3,4 5, 6, 7, 8, 9, 10 or more microchannels.
10. The microfluidic device of any of paragraphs 1-9, wherein width of the at least one microchannel is from about 0.1 mm to about 10 mm.
11. The microfluidic device of any of paragraphs 1-10, wherein depth of the at least one microchannel is from about 100 μm to about 2000 μm.
12. The microfluidic device of any of paragraphs 1-11, wherein at least one of the microchannel comprises a plurality of grooves extending transverse to the channel in the capture chamber.
13. The microfluidic device of paragraph 12, wherein width of at least one of the groove is from about 0.1 μm to about 1000 μm.
14. The microfluidic device of any of paragraphs 12-13, wherein depth of at least one of the grooves is from about 0.1 μm to about 500 μm.
15. The microfluidic device of any of paragraphs 12-14, wherein space between the grooves is from about 0.1 μm to about 1000 μm.
16. The microfluidic device of any of paragraphs 1-15, wherein the fluid source provides a source fluid containing target components bound to magnetic microbeads.
17. The microfluidic device of any of paragraphs 1-16, wherein the source fluid is a biological fluid selected from the group consisting of blood, plasma, serum, lactation products, amniotic fluids, sputum, saliva, urine, semen, cerebrospinal fluid, bronchial aspirate, perspiration, mucus, liquefied stool sample, synovial fluid, lymphatic fluid, tears, tracheal aspirate, and any mixtures thereof.
18. The microfluidic device of any of paragraphs 1-17, wherein the source fluid is a non-biological fluid selected from the group consisting of water, organic solvents, saline solutions, sugar solutions, carbohydrate solutions, lipid solutions, nucleic acid solutions, hydrocarbons, acids, gasoline, petroleum, liquefied foods, gases, and any mixtures thereof.
19. The microfluidic device of any of paragraphs 1-18, wherein the target component is selected from the group consisting of hormones, cytokines, proteins, peptides, prions, lectins, oligonucleotides, molecular or chemical toxins, and any combination thereof.
20. The microfluidic device of any of paragraphs 1-19, wherein the target component is a bioparticle/pathogen selected from the group consisting of living or dead cells (prokaryotic and eukaryotic, including mammalian), viruses, bacteria, fungi, yeast, protozoan, microbes, parasites, and the like.
21. The microfluidic device of paragraph 20, wherein the target component is a cell selected from the group consisting of stem cells, cancer cells, progenitor cells, immune cells, blood cells, fetal cells, and the like.
22. The microfluidic device of any of paragraphs 1-21, wherein the microfluidic device is fabricated from a biocompatible material.
23. The microfluidic device of any of paragraphs 1-22, wherein the microfluidic device is fabricated from a material selected from the group consisting of
   polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.
24. The microfluidic device of any of paragraphs 1-23, wherein the source fluid flows at a rate of 1 mL/hr to 1000 L/hr through the microchannel.
25. The microfluidic device of any of paragraphs 1-24, further comprising a micromolded reservoir with a channel connected to the capture chamber.
26. The microfluidic device of any of paragraphs 1-25, wherein the magnetic microbead is from about 1 nm to about 1 mm in size.
27. A method of identifying at least one target component in a source fluid comprising:
   mixing a plurality of magnetic microbeads with the source fluid to enable binding of at least one target component to one or more magnetic microbeads, wherein a surface of the magnetic microbeads is functionalized to include at least one binding molecule that can bind with the target component in the fluid;
   flowing the source fluid through a microdevice of any of paragraphs 1-26;
   exposing the source fluid containing at least one magnetic microbead bound target component to a magnetic field gradient positioned to cause the magnetic microbead bound target component to migrate into the capture chamber; and
   detecting and/or analyzing at least one of the magnetic microbead target components in the capture chamber.
28. The method of paragraph 27, further comprising pretreating the source fluid before mixing with the magnetic microbeads.
29. The method of any of paragraphs 27-28, wherein from about 10 to about 10⁶ of magnetic microbeads are mixed with 1 ml of the source fluid.
30. The method of any of paragraphs 27-29, wherein the source fluid is from 1 ml to about 500 ml.
31. The method of any of paragraphs 27-30, wherein the source fluid flow rate through the microchannel is from about 1 ml/hr to 1000 L/hr.
32. The method of any of paragraphs 27-31, wherein detecting and/or analyzing at least one of the magnetic microbead bound target components in the capture chamber includes viewing the target components under a microscope.
33. The method of any of paragraphs 27-32, wherein detecting and/or analyzing at least one of the magnetic microbead bound target components in the capture chamber includes labeling the target component with a label.
34. The method of paragraph 33, wherein the label is selected from the group consisting of fluorescent molecules, radioisotopes, nucleotide chromophore, enzymes, substrates, chemiluminescent moieties, magnetic microbeads, bioluminescent moieties, and the like.
35. The method of any of paragraphs 33-34, wherein the label is a fluorescent label.
36. The method of any of paragraphs 33-35, wherein the label is a fluorescent label selected from the group consisting of Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.
37. The method of any of paragraphs 33-36, wherein said labeling comprising flowing a fluid comprising a labeling molecule through the capture chamber.
38. The method of any of paragraphs 33-37, wherein said labeling comprising flowing a fluid comprising a first labeling molecule and a second labeling molecule through the capture chamber.
39. The method of any of paragraphs 27-38, further comprising washing the microchannel before said detecting and/or analyzing.
40. The method of paragraph 39, wherein said washing comprising flowing a fluid through the microchannel.
41. The method of paragraph 40, wherein said fluid is a buffer.
42. The method of any of paragraphs 39-41, wherein said fluid is from 0.5× to about 10× volume of the source fluid.
43. The method of any of paragraphs 39-42, wherein said fluid is from 0.5× to about 10× total volume of the microchannels.
44. The method of any of paragraphs 27-43, wherein source fluid is a biological fluid selected from the group consisting of blood, plasma, serum, lactation products, amniotic fluids, sputum, saliva, urine, semen, cerebrospinal fluid, bronchial aspirate, perspiration, mucus, liquefied stool sample, synovial fluid, lymphatic fluid, tears, tracheal aspirate, and any mixtures thereof.
45. The method of any of paragraphs 27-43, wherein the source fluid is a non-biological fluid selected from the group consisting of water, organic solvents, saline solutions, sugar solutions, carbohydrate solutions, lipid solutions, nucleic acid solutions, hydrocarbons, acids, gasoline, petroleum, liquefied foods, gases, and any mixtures thereof.
46. The method of any of paragraphs 27-45, wherein the target component is selected from the group consisting of hormones, cytokines, proteins, peptides, prions, lectins, oligonucleotides, molecular or chemical toxins, and any combination thereof.
47. The method of any of paragraphs 27-46, wherein the target component is a bioparticle/pathogen selected from the group consisting of living or dead cells (prokaryotic and eukaryotic, including mammalian), viruses, bacteria, fungi, yeast, protozoan, microbes, parasites, and the like.
48. The method of paragraph 47, wherein the target component is a cell selected from the group consisting of stem cells, cancer cells, progenitor cells, immune cells, blood cells, fetal cells, and the like.
49. The microdevice of any of claims 1-26, wherein the magnetic microbead is a MBL coated magnetic microbead.
50. The method of any of claims 27-48, wherein the magnetic microbead is a MBL coated magnetic microbead.

To the extent not already indicated, it will be understood by those of ordinary skill in the art that any one of the various embodiments herein described and illustrated may be further modified to incorporate features shown in any of the other embodiments disclosed herein.

The following example illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following example does not in any way limit the invention.

### EXAMPLE

A prototype magnetic pathogen detection device was fabricated by plasma bonding a single layer of micromolded polydimethylsiloxane (PDMS) (60×25×3 mm; width (w)×length (l)×height (h)) to a microscope glass slide (60×24×0.167 mm; width (w)×length (l)×height (h)) (FIG. 10). This micromolded PDMS contains a single long channel (2.5 mm×4 cm×560 um; width (w)×length (l)×height (h)). The middle 20 mm of the channel length contains 100 um wide and 80 um deep grooves that repeat every 200 um, forming a regular washboard-like feature that comprises the ceiling of the capture chamber. The main channel feature was micromolded from a sticker-based mold manufactured by a cutter-plotter while the washboard feature was photolithographically produced using SU-8 molding.

The magnetic concentrator was micromachined from the permalloy EFI Alloy 79 (10 mm×25 mm×1.55 mm, width (w)×length (l)×height (h)) with the front 5 mm tapered to reduce the strength of magnetic field gradient followed by a repeating washboard of 400 um deep by 400 um long grooves that serve to angle and concentrate the magnetic field around them, giving us a more uniform distribution of magnetic force on the particles in the capture chamber (FIGS. 10B and 11). The permalloy flux concentrator is magnetized using a permanent neodynium magnet (NdFeB) (dimensions 0.75″×0.75″0.75″ width (w)×length (l)×height (h)). This combination creates a realtively uniform magnetic field along the length of the capture chamber in the PDMS channel with a higher magnetic field gradient than is possible with a permanent magnet alone (FIG. 11).

Magnetically tagged pathogens are pulled to the ceiling of the capture chamber by the magnetic field gradient where they settle into the washboard grooves, which shields them from the fluid flow and greatly reduces the fluidic drag they experience, preventing them from being swept downstream (FIG. 10B). The magnetic concentrator reinforces this by locally angling the magnetic field so that the force on the beads directly opposes the fluidic drag as well.

In one example, a 10 mL blood sample was first treated with 10 u/ml heparin to prevent coagulation during the assay and 1% by volume Triton X-100 to selectively lyse the majority of mammalian cells in the sample, taking advantage of the more robust cell walls present on fungi and bacteria. The lysis step speeds up the binding and simplifes the the fluidic handling of the blood by reducing its non-Newtonian and coilloidal fluid properties. An anti-phagocytotic temperature shock procedure shuts down any remaining white blood cells and prevents them from phagocytoszying the micromagnetic beads. Immediately after the 15 minute sample preparation procedure, the MBL beads coated with opsonins (e.g., antibodies, Mannose Binding Lectin) were added to the sample where they bind specifically to the pathogens, creating a magnetic handle on the cells of interest that can be exploited to rapidly isolate and concentrate the pathogens from the blood sample.

After the entire blood sample has been run through the device, a saline dye solution containing two dyes was run through the device to fluorescently tag the cells. One dye was calcofluor (1 μM to 100 μM)—a bright, fast-acting dye that adheres to chitin moities present in the cell walls of pathogenic fungi. The second stain was more specific, using a primary antibody produced and purified from an in-house scFv phage display library to identify the genus and/or species of the captured pathogens. The staining buffer was followed by a saline wash to remove excess dye from the channel before imaging. The use of two stains provides more specificity relative to using a single dye only. The fast, reliable and bright calcofluor (1 μM to 100 μM) stain allowed us to quickly identify potential fungi in the sample while the second, more targeted stain allowed us to confirm that the labeled cell was an actual pathogen rather than background noise. The use of the second stain can also provide more specific information on the captured pathogen depending on the specificity of the antibody used.

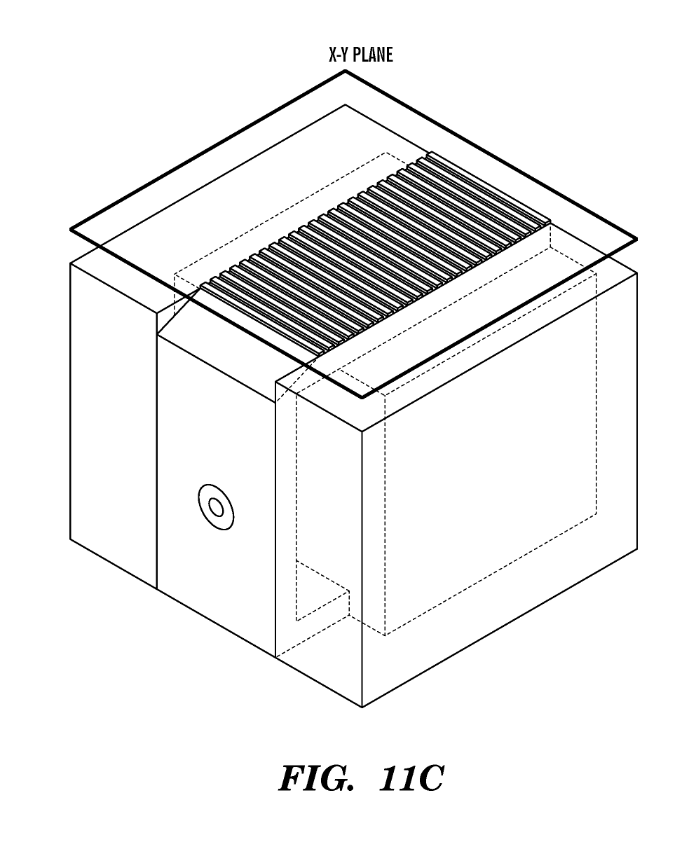

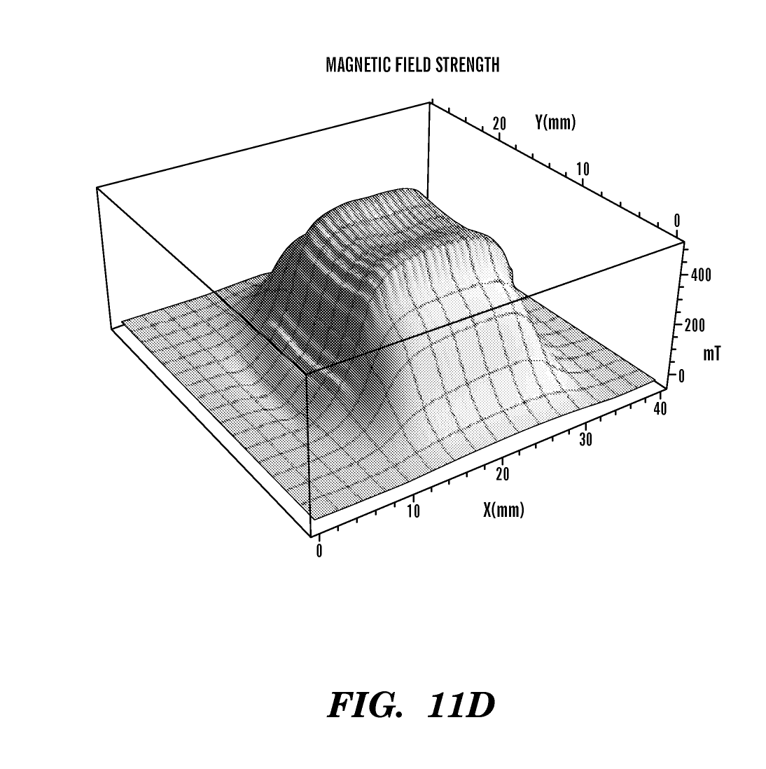

The stains were then visualized using an epifluroescent microscope to indentify pathogen. Results are shown in FIGS. 12 and 13.

### Discussion

Our current prototype diagnostic device and procedure is able to detect as few as twenty C. albicans fungal pathogens in 10 mL samples of human whole blood spiked with 2 cell colony forming units (cfu)/mL within ˜60 min after sample collection (FIG. 12A). Control experiments in which the magnetic beads were attached to the fungi before they were spiked into whole blood (FIG. 12) revealed that the device actually has a detection threshold as low as 0.4 cfu/ml or better (as few as 4 fungi per 10 ml). Improvements in bead spreading have increased the sensitivity of the device more than 5 times over our original prototype as disclosed in U.S. Prov. App. No. 61/296,355, filed Jan. 19, 2010. Live C. albicans cells were bound by magnetic beads (1 um diameter) that were pre-coated with MBL that binds to sugar groups (e.g. mannan) found on the surface of these cells, and then they were magnetically captured in our diagnostic device. The captured cells were fluorescently labeled by flowing the cellulose-binding calcofluor (1 μm to 100 μM) dye and a secondary immunostain through the microfluidic channel, making them easily distinguishable among the many non-fluorescent beads using a conventional inverted epifluorescent microscope (DAPI and FITC filter cubes; 200× magnification). Using this approach, we could demonstrate a direct correlation between the number of fungal cells identified with the micromagnetic-microfluidic diagnostic device and the concentration of pathogenic cells in the blood samples analyzed (because, in this case, known amounts of fungal cells were added to the blood) (FIG. 12A). These data demonstrate the usefulness of this method for rapid (<60 min) diagnosis of blood-borne fungal infection, as well as quantification of the fungal pathogen load in human blood samples.

Narrowing the width of the channel to a single 2.5 mm in subsequent studies made it possible to image the entire capture chamber with one scan along the length of the channel, greatly decreasing the required counting time. Increasing the height of the channel compensated for decreasing its width, allowing us to maintain a low average fluid velocity despite having a high flow rate of blood through the device.

Balancing the magnetic force on the tagged cells created by the applied magnetic field and the Stokes drag force on the cells provides an estimate of the velocity and direction of cell movement inside the device,

$$v ∝ \frac{n ∇ ( m \cdot B )}{6 nr {μμ}_{o}} ,$$

where v is the velocity of the tagged cell, n is the number of magnetic beads bound to the cell, m is the magnetic dipole of a single bead, B is the magnetic field created in the channel, r is the diameter of the pathogen, μ is the approximate viscosity of blood and μₒ is magnetic permeability of vacuum.

The trajectory of each magnetically tagged cell can be estimated from this equation, and the channel dimensions and fluidic flow rate can be adjusted to ensure that more than 99% of the magnetic particles will be retained in the capture chamber.

The addition of washboard-like features to the ceiling of the capture chamber creates small pockets where the beads are sheltered from the fluidic drag forces that would tend to push them downstream, causing them either to form a dense pile at the end of the capture chamber or be swept out of the device. Instead the magnetic beads and tagged cells stay where they were pulled to the ceiling of the capture chamber when they settle into the washboard, making it unnecessary to rearrange them for counting and increasing the number of captured cells that can be seen for rapid pathogen quantitation.

Both computer simulations and experimental results showed that the neodymium magnets that were used had the highest magnetic field gradients at the leading and trailing edge of the magnets, causing the majority of magnetic particles to arrest at these sites rather than uniformly distributing along the length of the channel (FIG. 11A). To overcome this limitation, we created a magnetic flux concentrator to disperse the magnetic field at the leading edge of the magnet and focus it along the length of the chamber, creating a much more uniform magnetic field and giving a much better distribution of magnetic beads during capture in the device. The 400 um teeth machined into the concentrator act to create a high field gradient locally at the surface of each ‘tooth’ and to angle the magnetic field so that the force exerted on the beads and tagged cells can directly oppose the fluidic drag on them in the capture chamber. This, combined with the micropatterning in the capture chamber, gives us a much more uniform spread of the magnetic particles than was possible with previous designs and greatly facilitates quantification of captured cells (FIG. 13). Th

The use of two stains to identify captured cells greatly improved the specificity of the assay by allowing us to definitively differentiate between non-specific background staining and actual pathogens. Control experiments testing this method were carried out using GFP-transfected C. albicans spiked blood samples that were then also stained with calcolfuor, although primary or secondary antibody staining can replace the GFP transfection without sacrificing any sensitivity.

The use of MBL coated micromagnetic beads or engineered Opsonin (as disclosed in U.S. Prov. App. No. 61/296,222, filed Jan. 19, 2010) allows this assay to be used to capture and identify a large range of pathogenic organisms in whole blood without any foreknowledge of infectious organism. This is not possible when using antibody coated beads, and it is a critical requirement for clinical use where the infectious pathogen is not known prior to testing and evaluation. The MBL beads can be added to a blood sample where they specifically bind to the pathogenic cells, providing a way to differentiate them from the rest of the cells in blood so that they can be separated out and imaged using the microfluidic capture device. Sensitivity and specificity are provided by the staining and quantification procedures used to count the captured cells. While, the bead size and staining was optimized for fungi in this example, small modifications can make this diagnostic technology equally effective for identifying bacteria, protozoa and even viruses from whole blood due to the broad binding characteristics of mannose binding lectin.

Improvements in the design of the capture chamber and the shape of the magnetic field have greatly increased the capture and visualization efficiency in whole blood over our original prototypes described in U.S. Provisional Application No. 61/296,355, filed Jan. 19, 2010, by 5 to 6 times, allowing us to recover and quantify as few as 20 fungi in whole blood, and less than 1 fungal cell per ml in some studies. Addition of size based separation schemes to remove excess beads from the capture chamber can further increase in the sensitivity of this assay to well below 1 cfu/ml.

Beyond the specific application of detecting fungi in blood, this diagnostics technology can be used for the general purpose of selectively detecting very low concentrations of any pathogenic cells, viruses and molecules or infected cells from various fluids that are otherwise undetectable or require time-consuming culture, analysis or bioassays to detect. And thus, this diagnostic technology is a platform that will enable the rapid detection and diagnosis of wide variety of diseases, where each diagnosis is customized based on the use of opsonins that are specific to that disease. Opsonins used to bind specific particles of interest may include antibodies, as well as protein- or nucleotide-based aptamers and antigen binding proteins (such as MBL). Techniques such as directed evolution and phage display can be used to further optimize specificity and strength of particle binding.

The invention provides a simple, rapid and highly sensitive microfluidic-microdevice for pathogen detection that has a significant value as a POC diagnostic, as well as a rapid detection and pathogen collection device in the hospital setting. We demonstrated the utility of this device using living C. albicans pathogens that are a major cause of sepsis in humans, and we accomplished this in whole human blood without requiring any pre-processing of blood. The high sensitivity of this method using simple fluorescent stains is amenable to conventional fluorescent microscopes or LED detectors that may be integrated on chip within these microdevices enables us to detect less than 1 cfu/mL of blood. As patients with systemic blood borne infections usually have greater than 1 cfu/mL in their blood, this method be used with less than 10 mL of human blood, which is easily accommodated for POC applications. Although we used fluorescent labels and microscopic detection to demonstrate the feasibility of this approach, alternative and even more sensitive detection components, such as optical resonators that can detect single, unlabelled viral particles (Vollmer, F., Arnold, S. & Keng, D. Proc. Nat. Acad. Sci. USA, 2008, 105:20701-20704) or electrochemical detectors, can be integrated into these devices so that microscopes are not required. These microsystems devices also can be easily sterilized and disposed of after use to minimize potential infection. Due to their low cost of fabrication, simplicity of use, high sensitivity, and ability to isolate living pathogens that can be inserted into existing pathogen culture and sensitivity assays, these microdevice may therefore have wide spread value as first stage pathogen diagnostics in both the community and hospital settings.

### REFERENCES

1. Guery, B. P. et al. Management of invasive candidiasis and candidemia in adult non-neutropenic intensive care unit patients: Part I. Epidemiology and diagnosis. Intensive care medicine 35, 55-62 (2009).
2. Vollmer, F., Arnold, S. & Keng, D. Single virus detection from the reactive shift of a whispering-gallery mode. Proceedings of the National Academy of Sciences of the United States of America 105, 20701-20704 (2008).
3. Harry E. Burke. Burke, H. E. Handbook of magnetic phenomena. (Van Nostrand Reinhold, New York; 1986).
4. Pamme, N. Magnetism and microfluidics. Lab on a chip 6, 24-38 (2006).
5. M. Golosovsky, Y. S., D. Davidov Energy and symmetry of self-assembled two-dimensional dipole clusters in magnetic confinement. Physical Review E 65 (2002).
6. Weijia Wen, L. Z., and Ping Sheng Planar Magnetic Colloidal Crystals. Physical Review Letters 85 (2000).
7. Alsberg, E., Feinstein, E., Joy, M. P., Prentiss, M. & Ingber, D. E. Magnetically-guided self-assembly of fibrin matrices with ordered nano-scale structure for tissue engineering. Tissue engineering 12, 3247-3256 (2006).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A microfluidic device comprising:
an inlet port adapted to be connected to a fluid source;
an outlet port adapted to be connected to a fluid receiver;
at least one microchannel connected to and extending between the inlet port and the outlet port;
a capture chamber connected to the microfluidic channel, the capture chamber including at least one feature adapted to capture target components flowing in a source fluid provided by the fluid source; and
a magnetic source above the microchannel and configured to apply a magnetic field gradient to the source fluid flowing through the microchannel and to cause magnetic microbead bound target components in the source fluid to migrate into the capture chamber.

2. The microfluidic device of claim 1, wherein the microfluidic device further comprising a magnetic concentrator between the magnetic source and the microchannel.

3. (canceled)

4. The microfluidic device of claim 2, wherein the magnetic concentrator comprises a plurality of grooves on the surface adjacent to the microchannel.

5. The microfluidic device of claim 4, wherein width of at least one groove is from about 10 μm to about 1000 μm.

6. The microfluidic device of claim 4, wherein depth of at least one groove is from about 10 μm to about 2000 μm.

7. The microfluidic device of claim 4, wherein space between the grooves is from about 10 μm to about 1000 μm.

8. (canceled)

9. (canceled)

10. The microfluidic device of claim 1, wherein width of the at least one microchannel is from about 0.1 mm to about 10 mm.

11. The microfluidic device of claim 1, wherein depth of the at least one microchannel is from about 50 μm to about 2000 μm.

12. The microfluidic device of claim 1, wherein at least one of the microchannel comprises a plurality of grooves extending transverse to the channel in the capture chamber.

13. The microfluidic device of claim 12, wherein width of at least one of the groove is from about 0.1 μm to about 1000 μm.

14. The microfluidic device of claim 12, wherein depth of at least one of the grooves is from about 0.1 μm to about 500 μm.

15. The microfluidic device of claim 12, wherein space between the grooves is from about 0.1 μm to about 1000 μm.

16. (canceled)

17. The microfluidic device of claim 1, wherein the source fluid is a biological fluid selected from the group consisting of blood, plasma, serum, lactation products, amniotic fluids, sputum, saliva, urine, semen, cerebrospinal fluid, bronchial aspirate, perspiration, mucus, liquefied stool sample, synovial fluid, lymphatic fluid, tears, tracheal aspirate, and any mixtures thereof or the source fluid is a non-biological fluid selected from the group consisting of water, organic solvents, saline solutions, sugar solutions, carbohydrate solutions, lipid solutions, nucleic acid solutions, hydrocarbons, acids, gasoline, petroleum, liquefied foods, gases, and any mixtures thereof.

18. (canceled)

19. The microfluidic device of claim 1, wherein the target component is selected from the group consisting of hormones, cytokines, proteins, peptides, prions, lectins, oligonucleotides, molecular or chemical toxins, and any combination thereof or the target component is a bioparticle/pathogen selected from the group consisting of living or dead cells (prokaryotic and eukaryotic, including mammalian), viruses, bacteria, fungi, yeast, protozoan, microbes, parasites, and the like.

20. (canceled)

21. The microfluidic device of claim 19, wherein the target component is a cell selected from the group consisting of stem cells, cancer cells, progenitor cells, immune cells, blood cells, fetal cells, and the like.

22. The microfluidic device of claim 1, wherein the microfluidic device is fabricated from a biocompatible material.

23. (canceled)

24. (canceled)

25. The microfluidic device of claim 1, further comprising a micromolded reservoir with a channel connected to the capture chamber.

26. The microfluidic device of claim 1, wherein the magnetic microbead is from about 1 nm to about 1 mm in size.

27. A method of identifying at least one target component in a source fluid comprising:
mixing a plurality of magnetic microbeads with the source fluid to enable binding of at least one target component to one or more magnetic microbeads, wherein a surface of the magnetic microbeads is functionalized to include at least one binding molecule that can bind with the target component in the fluid;
flowing the source fluid through a microdevice of claim 1;
exposing the source fluid containing at least one magnetic microbead bound target component to a magnetic field gradient positioned to cause the magnetic microbead bound target components to migrate into the capture chamber; and
detecting and/or analyzing at least one of the magnetic microbead target components in the capture chamber.

28.-48. (canceled)

49. The microfluidic device of claim 1, wherein the magnetic microbead is a mannose binding lection (MBL) coated magnetic microbead.

50. (canceled)
